(19) United States
(12) Patent Application Publication
(10) Pub. No.: US 2024/0382425 A1
(43) Pub. Date: Nov. 21, 2024

(54) RNA binding and stabilising cationic liposome, its application and method of loading the liposome with emetine

(71) Applicant: BS BIOTECHNA SPOLKA Z OGRANICZONA ODPOWIEDZIALNOSCIA, Krakow (PL)

(72) Inventor: Olga SWIECH, Bieniewice (PL)

(21) Appl. No.: 18/683,763

(22) PCT Filed: Aug. 15, 2022
(86) PCT No.: PCT/PL2022/000046

§ 371 Date: Feb. 14, 2024

(30) Foreign Application Priority Data
Aug. 14, 2021 (PL) P.438742

(51) Int. Cl. A61K 9/127; A61K 31/4725; A61K 47/54; A61K 47/69; C12N 15/113

(52) U.S. Cl. CPC A61K 9/1272; A61K 9/1277; A61K 31/4725; A61K 47/545; A61K 47/6911; C12N 15/113; C12N 2310/14; C12N 2310/51

(57) ABSTRACT

The present invention relates to a cationic liposome that binds and stabilises RNA, its use and a method for loading the liposome with emetine. The liposome consists of neutral lipids in the amount of 12.4 to 49% by weight, cationic lipids in the amount of 16.2 to 55% by weight, polyethylene glycol-modified lipids in the amount of 12.9 to 15.1% by weight and cholesterol in an amount from 15.4 to 18.1% by weight, and is characterised by a size from 80 nm to 190 nm, a polydispersity index from 0.06 to 0.23, and a zeta potential from +19 mV to +55 mV, wherein it is loaded with RNA.

The present invention relates to a cationic liposome that binds and stabilises RNA, its use and a method for loading the liposome with emetine.

Gene therapy is defined as the use of different types of nucleic acids to express, edit, or silence specific genes in cells in order to achieve specific therapeutic effects. The first nucleic acid sequences used in gene therapy were in the form of plasmid DNA, while the recently introduced RNA-based drugs, such as small interfering RNA (siRNA) or mRNA, are characterised by much higher effectiveness. Despite the promising results of RNA therapy as a tool for the treatment of currently incurable genetic and acquired diseases, its use faces a number of difficulties, such as the toxicity of viral vectors utilised to deliver RNA and the ineffectiveness of non-viral or synthetic carriers to replace viral carriers. (Lukashev A. N., Zamyatnin A. A. Jr.: Viral vectors for gene therapy: current state and clinical perspectives. Biochemistry (Mosc.), 81, 700-708, 2016). The ineffectiveness of non-viral systems can be explained by the absence of efficient delivery systems that sufficiently protect the nucleic acids and deliver them at sufficient doses to target sites, usually specific cells. (Yin H., Kanasty R. L., Eltoukhy A. A., Vegas A. J., Dorkin J. R., Anderson D. G.: Non-viral vectors for gene-based therapy. Nat. Rev. Genet., 15, 541-555, 2014). Over the last few years, there has been tremendous progress in the development of non-viral systems for the in vivo targeted delivery of genes to various organs. This progress led to the approval of the first drug based on RNA interference (RNAi), Patisiran (Onpattro), for the treatment of transthyretin-mediated hereditary amyloidosis. (Kristen A V, Ajroud-Driss S, Conceicao I, Gorevic P, Kyriakides T, Obici L. Patisiran, an RNAi therapeutic for the treatment of hereditary transthyretin-mediated amyloidosis. Neurodegenerative Disease Management, 9, 5-23, 2019). Patisiran is a lipid-based formulation that contains siRNA in capsules and has reliable effect on liver hepatocytes when administered intravenously to effectively and permanently silence the abnormal form of the transthyretin gene. This breakthrough in the field of non-viral gene delivery has met with great interest worldwide and has clearly demonstrated the importance of lipid-based systems in developing more efficient drugs in the future.

Several synthetic vectors for gene delivery are available. One strategy is to use conjugates of different nucleic acids with different functional devices, such as peptides, polymers, sugars, proteins, antibodies or aptamers. (Glebova K V, Marakhonov A V, Baranova A V, Skoblov M. Types of non-viral delivery systems of small interfering RNA. Molekuliarnaia Biologiia, 46, 387-401, 2012). Another strategy is to encapsulate nucleic acids into appropriately sized nanoparticles (NPs). The conjugate systems are small in size and can be easily removed from the body by glomerular filtration in the kidney. (Khalil I A, Yamada Y, Harashima H. Optimization of siRNA delivery to target sites: issues and future directions. Expert Opin. Drug Deliv., 15, 1053-1065, 2018). Furthermore, the nucleic acids in the conjugates are unprotected and must be chemically modified to resist degradation by circulating nucleases. NP systems, on the other hand, are large enough to avoid renal elimination and can provide greater protection for circulating nucleic acids. The nanoparticles used for gene delivery can be broadly classified into polymeric and lipid nanoparticles. Lipid nanoparticles provide several benefits including higher stability, low toxicity, and greater efficiency. (Cullis P R, Hope M J. Lipid nanoparticle systems for enabling gene therapies. Mol. Ther., 25, 1467-1475, 2017; Zhao Y, Huang L. Lipid nanoparticles for gene delivery. Adv. Genet., 88, 13-36, 2014). In addition, lipid-based systems can be modified with other ligands that target specific cells. To increase efficiency and reduce the side effects of lipid-based gene carriers, systems must avoid serum inactivation and must be able to bind and stabilise nucleotides and target specific cells of the body. (Khalil I A, Sato Y, Harashima H. Recent advances in the targeting of systemically administered non-viral gene delivery systems. Expert Opin. Drug Deliv., 16, 1037-1050, 2019; Nakamura T, Yamada Y, Sato Y, Khalil I A, Harashima H. Innovative nanotechnologies for enhancing nucleic acids/gene therapy: controlling intracellular trafficking to targeted biodistribution. Biomaterials, 218, 119329, 2019). The lipid composition of nanoparticles determines their physicochemical parameters, durability, strength of nucleic acid binding and life time in the bloodstream. The most frequently used lipids for the construction of lipid carriers in gene therapy include: DOTMA, DOTAP and their derivatives, as well as ionizable lipids, their combinations with neutral lipids and polyethylene glycol-modified lipids.

Emetine is a small-molecule drug that exhibits both anti-tumour and anti-parasitic activity, and a strong broad-spectrum inhibitory effect against various DNA and RNA viruses. It can prevent viruses from entering cells, thereby inhibiting viral replication enzyme activity and intracellular transport, and can also inhibit the translation of viral proteins. (Mukhopadhyay R, Roy S, Venkatadri R, et al. Efficacy and mechanism of action of low dose emetine against human cytomegalovirus. PLOS Pathog 2016;12:e1005717). It has been confirmed that inhibition of protein translation by emetine may be effective in preventing replication of the severe acute respiratory syndrome coronavirus 2 (SARS-CoV-2) (Bojkova D, Klann K, Widera M, et al. Proteomics of SARS-CoV-2-infected host cells reveals therapy targets. Nature 2020;583:469-472.) In addition, among over 290 drugs that have been tested for the MERS and SARS coronaviruses, emetine has the lowest EC50 (less than 0.02 μM), blocking viral invasion and inhibiting viral replication. (Bleasel M D, Peterson G M. Emetine, Ipecac, Ipecac Alkaloids and Analogues as Potential Antiviral Agents for Coronaviruses. Pharmaceuticals (Basel) 2020;13:51). Additionally, it can accumulate in pulmonary tissue for more than 12 hours and still maintain an effective concentration. Because of that, emetine can be used as a potential COVID-19 drug. (doi: 10.1097/JBR.0000000000000076).

On the other hand, the anti-tumour effects of emetine have been known since the beginning of the 20^{th} century. In the 1970s, emetine was tested in Phase I and II clinical trials as an anticancer agent alone or in combination with other chemotherapy treatments. These studies have been performed in patients with advanced disease resistant to treatment, and partial and occasional complete response has been reported in pre-treated patients. Nevertheless, the therapeutic index of emetine was found to be very narrow and dose escalation was limited by the development of cardiac toxicity and ECG abnormalities. Unlike intraoral emetine, parenteral emetine did not cause significant nausea. Moreover, the drug had no effect on kidney or liver function and was not myelosuppressive. However, dose-limiting cardiac and muscle toxicity appeared to inhibit the further development of emetine as an anticancer therapy. These studies suggest that emetine may be a useful anticancer agent if its therapeutic index improves (EXCLI Journal 2016; 15:323-328—ISSN 1611-2156, published: May 10, 2016, Recent developments on potential new applications of emetine as anti-cancer agent November 2019, Volume 42 Issue 5, Emetine exhibits anticancer activity in breast cancer cells as an antagonist of Wnt/β-catenin signaling The Open Natural Products Journal, 2011, 4, 8-15, Biological Activities of Emetine).

The foregoing studies on emetine show that it can find potential use as an supporting agent in gene therapies directed against both viral and anticancer diseases. Therefore, there is a great need in the search for drug carriers ensuring safe transport of emetine to pathologically altered cells, in particular cationic carriers, ensuring both the binding and stabilization of ribonucleic acids. Previous studies have shown that emetine has a low affinity for lipid carriers with an encapsulation efficiency of % EE of 50% for neutral lipids, and its loading into lipid carriers was carried out using a concentration gradient in a separate synthetic step after the liposome formation process (DOI: 10.1016/j.ejpb.2014.04.002).

The essence of the solution according to the first invention consists in the fact that the liposome consists of neutral lipids in the amount of 12.4 to 49% by weight, cationic lipids in the amount of 16.2 to 55% by weight, polyethylene glycol-modified lipids in the amount of 12.9 to 15.1% by weight and cholesterol in an amount from 15.4 to 18.1% by weight, and is characterised by a size from 80 nm to 190 nm, a polydispersity index from 0.06 to 0.23, and a zeta potential from +19 mV to +55 mV, wherein it is loaded with RNA.

Preferably, the RNA is in the form of an anti-EGFR siRNA duplex. Preferably, the neutral lipid is dipalmitoylphosphatidylcholine (DPPC). Preferably, the neutral lipid is 1,2-dimyristoyl-sn-glycero-3-phosphocholine (DMPC).

Preferably, the cationic lipid is 1,2-dimyristoyl-sn-glycero-3-ethylphosphocholine (14:0 EPC)

Preferably, the cationic lipid is 1,2-dipalmitoyl-sn-glycero-3-ethylphosphocholine (16:0 EPC).

In a preferred embodiment, the polyethylene glycol-modified lipid is 1,2-distearoyl-sn-glycero-3-phosphoethanolamine-N-[amino (polyethylene glycol)-2000] (DSPE-PEG-NH₂).

Preferably, the RNA is linked to the liposomes at a ratio of 1:30, that is, 1 mg of liposome contains no more than 1.34 μg of RNA.

Preferably, the liposome has an embedded low molecular weight drug. Most preferably, the small molecule drug is emetine in an amount from 0.5% by weight and not more than 8.5% by weight.

Preferably, a liposome is modified with the targeting agent.

Preferably, the targeting agent is folic acid in an amount of not less than 1.3% by weight and not more than 1.55% by weight.

The essence of the embodiment of the second invention is the use of the liposome described as a drug carrier above.

The liposome is preferably used in gene therapy.

The liposome is preferably used as a drug component in antiviral therapy. The liposome is preferably used as a drug component in anticancer therapy.

The essence of the solution according to the invention in terms of the method is that from 12.4 to 49% by weight of neutral lipids, from 16.2 to 55% by weight of cationic lipids, from 12.9 to 15.1% by weight of polyethylene glycol-modified lipids, cholesterol in an amount from 15.4 to 18.1% by weight, and not less than 0.5% by weight of emetine are weighed, and then the weighing amount is dissolved in a 2:1 mixture of chloroform and methanol, adding from 0.26 to 1.4 mg of lipid mixture for every 1 mL of solvent mixture. Then the entirety is evaporated, removing the solvent at a temperature of 25 to 40° C., at a pressure of 100 to 300 mbar for 2 to 5 hours, until a thin lipid bilayer film is obtained on the vessel walls, and then the film is post-dried at a temperature of 25° C. to 40° C., at a pressure of 100 to 300 mbar. After complete evaporation of the organic solvents, sterile filtered saline, PBS buffer or Ringer's solution is added in the amount of 1 mL per 0.34 to 0.36 mg of lipids, tightly closed and then hydrated at a temperature of 45 to 60° C. at a pressure of 100 up to 300 mbar for 5 to 10 hours. The hydrated lipid layers are allowed to cool and then the lipids are subjected to the process of breaking the lipids into particles not exceeding 200 nm at a temperature of 45 to 65° C., controlling the particle size and the amount of bound emetine. The solution is allowed to cool, finally the obtained liposomes are purified from unbound emetine and unreacted lipids, the Zeta potential is measured and the characterization of the composition is made. Preferably, the neutral lipid is dipalmitoylphosphatidylcholine (DPPC). Preferably, the neutral lipid is 1,2-dimyristoyl-sn-glycero-3-phosphocholine (DMPC).

Preferably, the cationic lipid is 1,2-dimyristoyl-sn-glycero-3-ethylphosphocholine (14:0 EPC).

Preferably, the cationic lipid is 1,2-dipalmitoyl-sn-glycero-3-ethylphosphocholine (16:0 EPC).

Preferably, the polyethylene glycol modified lipid is 1,2-distearoyl-sn-glycero-3-phosphoethanolamine-N-[amino (polyethylene glycol)-2000] (DSPE-PEG-NH₂).

Preferably, emetine is prepared as follows: prepare a 30 mg/mL solution of emetine hydrochloride in deionised water, then precipitate by titration with 0.2 M NaOH solution, 1 mL 0.2 M NaOH for every 20 mg of emetine, and centrifuge the precipitate at 4,000 to 6,000 RPM for 10 to 30 minutes, and separate it from the solution. 0.1 mL of 0.2 M NaOH is added to the supernatant solution to control the complete precipitation of the emetine form, then the amount of unprecipitated emetine is measured, and the precipitate is washed not less than five times with distilled water, each time dispersing with ultrasound, centrifuging and measuring the amount of unprecipitated emetine in the supernatant solution, the total loss of emetine during the process is not more than 20%. Next, the precipitate is post-dried at the temperature from 25 to 30° C. and pressure of 100 mbar for 3 to 5 hours.

Preferably, the amount of unprecipitated emetine is measured by UV-Vis spectroscopy against a calibration curve.

Preferably, the lipid mixture is evaporated in a vacuum dryer at a temperature of 25 to 40° C. and a pressure of 100 to 300 mbar for 2 to 4.5 h. Preferably, the lipid mixture is evaporated in a vacuum evaporator at a temperature of 30 to 40° C. and a pressure of 150 to 300 mbar for 2 to 5 hours.

Preferably, the hydration is carried out in a vacuum dryer.

Preferably, the hydration is performed in a vacuum evaporator.

Preferably, post-drying is carried out in a vacuum dryer.

Preferably, sterile saline, PBS buffer or Ringer's solution is filtered through bpPVDF, 0.2 μm syringe filters.

Preferably, the breakdown of lipids is carried out by sonication for 30 to 60 minutes at a frequency of 50 Hz until the colour of the solution changes from milky to colourless.

Preferably, the amount of bound emetine is monitored by the UV-Vis method at a wavelength of 280 nm.

Preferably, the liposomes are purified by dialysis on 10 kD membranes with ultrapure water for 30 to 60 minutes by adding 1 litre of water to 4.5 mL of sample and changing the water three times at regular intervals.

Preferably, the purity of the liposomes obtained is measured by UV-Vis spectroscopy.

Preferably, the size of the resulting liposomes is determined by the DLS method.

Preferably the characterization of the liposome composition is performed by the ATR IR method.

The main advantage of the solutions according to the inventions is that the combination of lipids used in the liposomes allows for the control of the positive surface charge and thus the control of the binding strength of RNA molecules and the stabilization of natural, unstable RNA strands, and their storage at +4° C. for up to 5 days, while commercially available RNA carriers require that the storage temperature is maintained at −80° C. The obtained liposomes bind RNA even at a weight ratio of 1:30 (RNA:liposome), 1 mg of liposomes can bind as much as 34 μg of nucleic acids. The developed liposomes are characterised by a small diameter and a low PDI polydispersity index.

Another advantage is the synthesis of cationic liposomes loaded with a low molecular weight drug—emetine with anti-tumour and antiviral properties for binding and stabilising RNA with a decorated targeting molecule—folic acid. The addition of emetine and folic acid in the structure of the cationic liposome supports the binding and stabilization of RNA. The obtained cationic liposomes containing emetine and folic acid show a marked selectivity in acting as carriers in anticancer therapy, they strongly penetrate and transport nucleic acids to Caco-2 tumour cells, which overexpress the folic acid receptor, while the effect on normal MEF-WT cells is negligible.

The obtained cationic liposomes without the targeting agent (folic acid) also efficiently transport RNA into the Caco-2 tumour cells, but their effect is weaker than in the case of cationic liposomes containing the targeting molecule-folic acid. However, they can be successfully used for anticancer gene therapy.

The procedure of preparing emetine-loaded liposomes allows to obtain cationic nanoparticles with emetine, with encapsulation efficiency % EE even greater than 90%, which was not achieved even for neutral liposomes. It also allows the reduction of synthetic steps and the loading of emetine during the formation of the lipid bilayer.

During the process of liposome formation according to the invention, an innovative combination of 16:0 EPC and 14:0 EPC cationic lipids with neutral lipids was used, and the synthesis of cationic liposomes loaded with a low molecular weight drug—emetine with anti-tumour and antiviral activity to bind and stabilise RNA, was obtained for the first time.

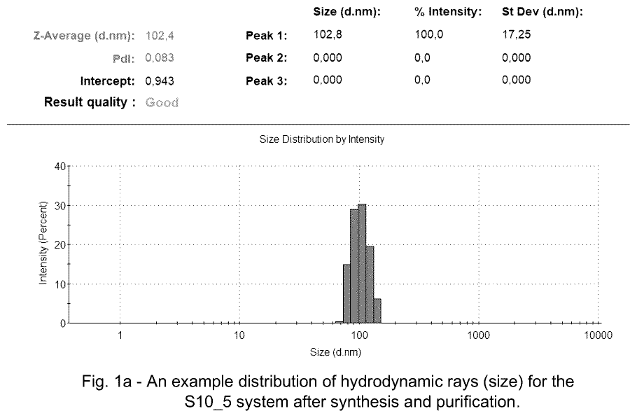

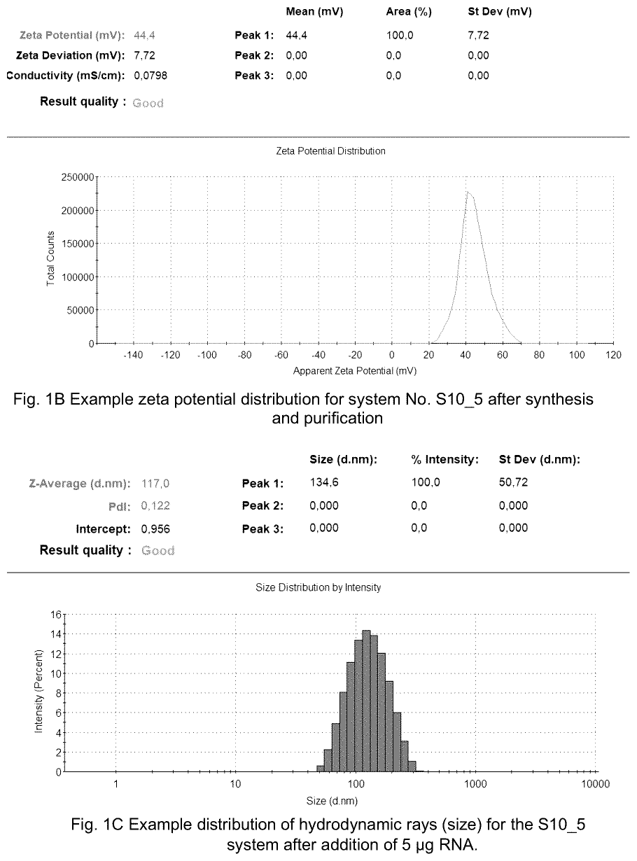

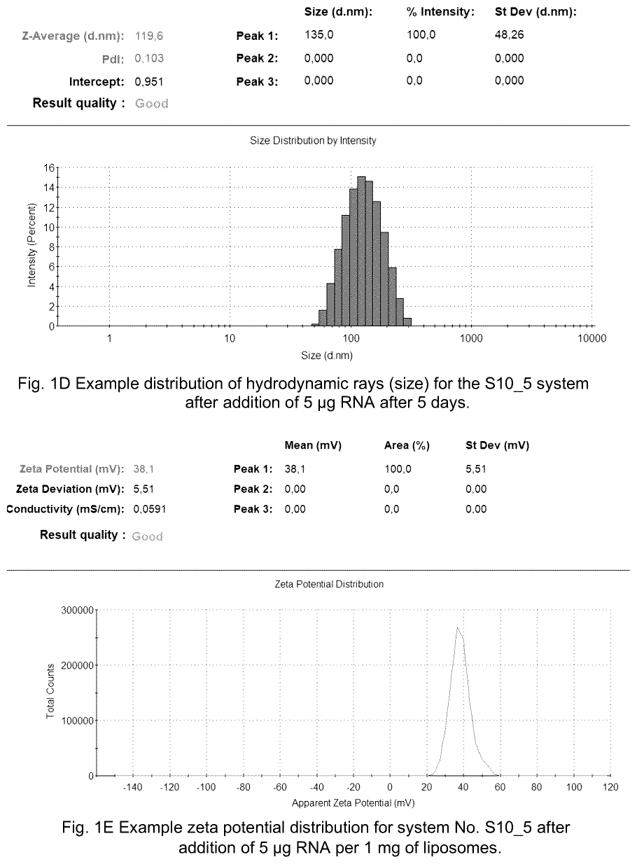

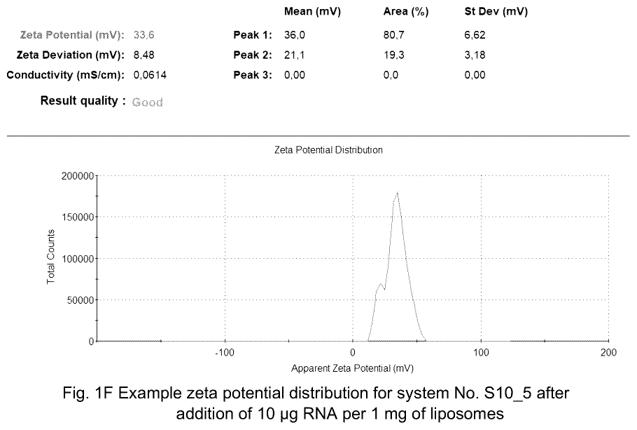

The solutions according to the invention are illustrated in the following embodiments and illustrated in FIG. 1-FIG. 16.

The tests used RNA, the characteristics of which are presented in the Table below:

| Synthesis yield | | | | | | |
|---|---|---|---|---|---|---|
| | | | | | in optical | |
| | | HPLC | | | units | Amount |
| Oligo- | | retention | | | Synthesis | at |
| nucleotide | | time | | | yield | μg/1 |
| symbol | 5′→3′ Sequence | Rt [min] | m/z_{calc} | m/z_{Ms} | [OD] | OD |
| RNAs | 5′-UCGG GCU CUG | 12.36 (B1) | 7,117.4 | 7,117.0 | 2 | 32.6 |
| | GAG GAA AAG | | | | | |
| | ATT-3′ | | | | | |
| | 22-nt | | | | | |
| RNAas | 5′-UCUU UUC CUC | 12.04 (B1) | 6,848.1 | 6,847.9 | 2 | 34.5 |
| | CAG AGC CCG | | | | | |
| | ATT-3′ | | | | | |
| | 22-nt | | | | | |
| DNA-FL | FL-5′-TTT CTT | 14.26 (B2) | 7,149.3 | 7,149.2 | 10 | 34.2 |
| | TTC CTC CAG | | | | | |
| | AGC CCGA-3′ | | | | | |
| | 22-nt | | | | | |
| RNA22s | 5′-UCGG GCU CUG | 12.55 (B1) | 7,167.4 | 7,167.0 | 19 | 31.6 |
| | GAG GAA AAG | | | | | |
| | AAA-3′ | | | | | |
| | 22-nt | | | | | |
| RNA-m1 | 5′-AUG CCC CUC | 11.82 (B1) | 8,341.9 | 8,341.0 | 20 | 34.3 |
| | AAC GUU UUU | | | | | |
| | CUU UUC CUC-3′ | | | | | |
| | 27-nt | | | | | |
| RNA-m2 | 5′-GAGG AGA CAU | 11.76 (B1) | 8,917.4 | 8,916.1 | 15 | 31.9 |
| | GGU GAA UUU | | | | | |
| | CUU UUC CUC-3′ | | | | | |
| | 28-nt | | | | | |
| RNA 2′OMe | 5′-2′OMe(AUG | 14.49 (B1) | 6,954.6 | 6,954.2 | 5 | 32.2 |
| (13b KBAC) | AAG GUUCAA | | | | | |
| | UCUNGAU UUU)-3′ | | | | | |
| | 21-nt | | | | | |

An anti-EGFR siRNA duplex was also used in a separate study. Cellular overexpression of EGFR has been associated with tumour malignancy, including tumour invasiveness, angiogenesis, and metastasis (Nicholson R I, Gee J M, Harper M E. EGFR and cancer prognosis. Eur J Cancer. 2001;374(suppl 4):9-15). Elevated levels of EGFR are found in various types of tumours, and overexpression of EGFR has been recognised as a prognostic marker of cancer progression and drug resistance in chemotherapy. Therefore, EGFR has been established as a therapeutic target in the treatment of cancer.

### Example I (Series I, Liposome S10_5 With RNA-m2)

The lipid masses appropriate for the series were weighed according to Table 1 (liposome 4, system code S10_5) in the amount of 10 times, i.e. 0.95 mg DMPC, 3.42 mg DPPC, 4.15 mg 14:0 EPC, 1.88 mg DSPE-PEG-NH2 and 2.25 mg of Cholesterol. It was then dissolved in 5 mL of a 2:1 volume ratio of chloroform with methanol. The lipid mixture was allowed to evaporate the solvents in a vacuum evaporator with the following parameters: T=40° C., p=200 mbar, t=3 h. A thin lipid bilayer film was formed during the evaporation process. The layer was dried in a vacuum dryer with the following parameters: T=40° C., p=200 mbar, t=1 h. After complete evaporation of the organic solvents, 45 mL of sterile saline filtered through bpPVDF, 0.2 μm syringe filters was added. The entirety was hydrated in a vacuum evaporator under the following conditions: T=60° C., p=300 mbar, t=5 h. Hydrated lipid layers were allowed to cool down and then sonicated in accordance with the parameters: T=60° C., t=40 min., 50 Hz. After this time, the colour of the solution changed from milky to colourless, which indicated the formation of small unilameller vesicles (SUVs). The liposome solution was then allowed to cool and their size was examined using the DLS dynamic light scattering method. The obtained liposomes were divided and purified by dialysis on 10 kD membranes with ultra pure water (Milli-Q system, Biopak CDUFBI001 filter: RNase free, DNase-free, Pyrogen-free) for 50 minutes (4.5 mL of sample per 1 L of water), changing the water three times after 20 minutes, 30 minutes and 40 minutes. The purity of the obtained liposomes was measured by UV-Vis spectroscopy. Zeta potential and size were then measured using the DLS method, and composition characterization was performed using the ATR IR method. The measurement results for the S10_5 liposomes and the remaining series I liposomes are presented in Table 2.

TABLE 1
Composition of series I liposomes expressed as molar, mass and percentage ratios.

| | | | | | | | | | | |
|---|---|---|---|---|---|---|---|---|---|---|
| | % by weight | | | | | | DSPE- | | DSPE- | |
| | of cationic | System | | | 16:0 | 14:0 | PEG- | Choles- | PEG- | |
| Liposomes | phospholipids | CODE | DMPC | DPPC | EPC | EPC | NH2 | terol | FA | Emetine |

| | | | |
|---|---|---|---|
| 1 | 23.74 | S9_5 | Molar ratio |

| 1.2 | 0.16 | 0.64 | 0 | 0.116 | 1 | 0 | 0 |
|---|---|---|---|---|---|---|---|
| Mass ratio [mg] | | | | | | | |
| 0.473 | 0.068 | 0.297 | 0 | 0.188 | 0.225 | 0 | 0 |
| Percentage by weight [%] | | | | | | | |
| 37.81 | 5.44 | 23.74 | 0.00 | 15.03 | 17.99 | 0.00 | 0.00 |

| | | | |
|---|---|---|---|
| 2 | 29.57 | S9_6 | Molar ratio |

| 0 | 0.8 | 0 | 1.2 | 0.116 | 1 | 0 | 0 |
|---|---|---|---|---|---|---|---|
| Mass ratio [mg] | | | | | | | |
| 0 | 0.342 | 0 | 0.518 | 0.188 | 0.225 | 0 | 0 |
| Percentage by weight [%] | | | | | | | |
| 0.00 | 26.87 | 0.00 | 40.69 | 14.77 | 17.67 | 0.00 | 0.00 |

| | | | |
|---|---|---|---|
| 3 | 24.78 | S10_4 | Molar ratio |

| 0.48 | 0.8 | 0 | 0.72 | 0.116 | 1 | 0 | 0 |
|---|---|---|---|---|---|---|---|
| Mass ratio [mg] | | | | | | | |
| 0.189 | 0.342 | 0 | 0.311 | 0.188 | 0.225 | 0 | 0 |
| Percentage by weight [%] | | | | | | | |
| 15.06 | 27.25 | 0.00 | 24.78 | 14.98 | 17.93 | 0.00 | 0.00 |

| | | | |
|---|---|---|---|
| 4 | 32.81 | S10-5 | Molar ratio |

| 0.24 | 0.8 | 0 | 0.96 | 0.116 | 1 | 0 | 0 |
|---|---|---|---|---|---|---|---|
| Mass ratio [mg] | | | | | | | |
| 0.095 | 0.342 | 0 | 0.415 | 0.188 | 0.225 | 0 | 0 |
| Percentage by weight [%] | | | | | | | |
| 7.51 | 27.04 | 0.00 | 32.81 | 14.86 | 17.79 | 0.00 | 0.00 |

| | | | |
|---|---|---|---|
| 5 | 40.69 | S10_6 | Molar ratio |

| 0 | 0.8 | 0 | 1.2 | 0.116 | 1 | 0 | 0 |
|---|---|---|---|---|---|---|---|
| Mass ratio [mg] | | | | | | | |
| 0 | 0.342 | 0 | 0.518 | 0.188 | 0.225 | 0 | 0 |
| Percentage by weight [%] | | | | | | | |
| 0.00 | 26.87 | 0.00 | 40.69 | 14.77 | 17.67 | 0.00 | 0.00 |

| | | | |
|---|---|---|---|
| 6 | 28.30 | S11_4 | Molar ratio |

| 0.72 | 0.48 | 0.32 | 0.48 | 0.116 | 1 | 0 | 0 |
|---|---|---|---|---|---|---|---|
| Mass ratio [mg] | | | | | | | |
| 0.284 | 0.205 | 0.149 | 0.207 | 0.188 | 0.225 | 0 | 0 |
| Percentage by weight [%] | | | | | | | |
| 22.58 | 16.30 | 11.84 | 16.45 | 14.94 | 17.89 | 0.00 | 0.00 |

| | | | |
|---|---|---|---|
| 7 | 41.95 | S11_5 | Molar ratio |

| 0.48 | 0.32 | 0.48 | 0.72 | 0.116 | 1 | 0 | 0 |
|---|---|---|---|---|---|---|---|
| Mass ratio [mg] | | | | | | | |
| 0.189 | 0.137 | 0.223 | 0.311 | 0.188 | 0.225 | 0 | 0 |
| Percentage by weight [%] | | | | | | | |
| 14.85 | 10.76 | 17.52 | 24.43 | 14.77 | 17.67 | 0.00 | 0.00 |

| | | | |
|---|---|---|---|
| 8 | 55.28 | S11_6 | Molar ratio |

| 0.24 | 0.16 | 0.64 | 0.96 | 0.116 | 1 | 0 | 0 |
|---|---|---|---|---|---|---|---|
| Mass ratio [mg] | | | | | | | |
| 0.095 | 0.068 | 0.297 | 0.415 | 0.188 | 0.225 | 0 | 0 |
| Percentage by weight [%] | | | | | | | |
| 7.38 | 5.28 | 23.06 | 32.22 | 14.60 | 17.47 | 0.00 | 0.00 |

TABLE 2
Physical parameters of series I liposomes
with standard deviations (SD).

| | | Physical parameters | | | | | |
|---|---|---|---|---|---|---|---|
| | | hydro- | | polydis- | | Zeta | |
| | | dynamic | | persity | | poten- | |
| | System | radius | SD | factor | SD | tial | SD |
| Liposome | CODE | [nm] | (+/−) | (PDI) | (+/−) | [mv] | (+/−) |
| 1 | S9_5 | 78.53 | 3.5 | 0.131 | 0.01 | 19.5 | 1.35 |
| 2 | S9_6 | 81.1 | 2.08 | 0.13 | 0.007 | 41.93 | 1.58 |
| 3 | S10_4 | 75.61 | 3.24 | 0.156 | 0.007 | 43.4 | 3.03 |
| 4 | S10_5 | 98.48 | 1.71 | 0.087 | 0.015 | 42.1 | 2.86 |
| 5 | S10_6 | 84.46 | 0.92 | 0.099 | 0.019 | 51.9 | 2.26 |
| 6 | S11_4 | 127.63 | 1.72 | 0.096 | 0.004 | 47.43 | 1.12 |
| 7 | S11_5 | 81.53 | 0.44 | 0.096 | 0.007 | 48.87 | 1.76 |
| 8 | S11_6 | 98.48 | 1.71 | 0.087 | 0.015 | 49.63 | 0.67 |

0.250 μL of RNA-m2 solution (20 μg/mL) dissolved in ultra pure water (Milli-Q system, Biopak CDUFBI001 filter: RNase free, DNase-free, Pyrogen-free), additionally sterilised twice in a medical steriliser, was added to 0.75 mL of S10_5 liposomes. The entirety was tightly closed, shielded from light sources and incubated at room temperature for 1 hour. After the incubation period, size and zeta potential were measured to exclude liposome aggregation and confirm RNA-m2 binding (zeta potential). Next, another 0.250 μL of RNA-m2 solution was added, the entirety was incubated for another hour, and the Zeta potential was measured to determine the RNA-m2 loading capacity of the liposomes.

The results are shown in Tables 3 and 4. The same experiments were performed for the remaining liposomes from series I (Table 1) and the results are presented in Tables 3 and 4. Additionally, measurements of the size of the systems were performed after 5 days to investigate the stability of the liposomes. The results are reported in Table 5.

TABLE 3
Size (hydrodynamic radius, HR) and polydispersity index (PDI) of liposomes
before and after the addition of 5 μg RNA-m2 per 1 mg of liposomes,
after one hour and five days with standard deviations (SD)

| | | | | | | After addition of | | | | 5 days after the | | | |
|---|---|---|---|---|---|---|---|---|---|---|---|---|---|
| | | | | | | 5 μg RNA per 1 | | | | addition of RNA 5 μg | | | |
| | | Before RNA addition | | | | mg of liposomes | | | | per 1 mg of liposomes | | | |
| | System | HR | SD | | SD | HR | SD | | SD | HR | SD | | SD |
| Item | CODE | [nm] | (+/−) | PDI | (+/−) | [nm] | (+/−) | PDI | (+/−) | [nm] | (+/−) | PDI | (+/−) |
| 1 | S9_5 | 78.5 | 3.5 | 0.131 | 0.01 | 78 | 1.2 | 0.141 | 0.011 | 80.8 | 1.7 | 0.144 | 0.008 |
| 2 | S9_6 | 81.1 | 2.1 | 0.13 | 0.007 | 82.4 | 0.8 | 0.116 | 0.007 | 80.8 | 1 | 0.126 | 0.012 |
| 3 | S10_4 | 75.6 | 3.2 | 0.156 | 0.007 | 82.6 | 1.7 | 0.135 | 0.012 | 83.6 | 1.2 | 0.124 | 0.017 |
| 4 | S10_5 | 98.5 | 1.7 | 0.087 | 0.015 | 106.1 | 1.7 | 0.128 | 0.006 | 136 | 1 | 0.11 | 0.008 |
| 5 | S10_6 | 84.5 | 0.9 | 0.099 | 0.019 | 90 | 0.9 | 0.099 | 0.014 | 90.8 | 0.6 | 0.092 | 0.007 |
| 6 | S11_4 | 127.6 | 1.7 | 0.096 | 0.004 | 137 | 2.6 | 0.108 | 0.026 | 138.1 | 1.5 | 0.12 | 0.01 |
| 7 | S11_5 | 81.5 | 0.4 | 0.096 | 0.007 | 90.8 | 0.8 | 0.085 | 0.007 | 92.6 | 0.8 | 0.092 | 0.001 |
| 8 | S11_6 | 98.5 | 1.7 | 0.087 | 0.015 | 100.9 | 0.5 | 0.068 | 0.017 | 101.4 | 1.2 | 0.07 | 0.013 |

The lack of significant changes in both the size and the polydispersity coefficient prove the lack of liposome aggregation, i.e. their stability in the presence of RNA-m2.

TABLE 4
Changes in the Zeta potential of series I liposomes after the addition of
5 μg and 10 μg RNA-m2 per 1 mg of liposomes with standard deviations (SD).

| | | | | After addition of 5 μg RNA- | | | After addition of 10 μg RNA- | | |
|---|---|---|---|---|---|---|---|---|---|
| | | | | m2 per 1 mg of liposomes | | | m2 per 1 mg of liposomes | | |
| | | Before the addition | | | | Zeta | | | Zeta |
| | | of RNA-m2 | | | | potential | | | potential |
| | System | Zeta | SD | Zeta | SD | change | Zeta | SD | change |
| No | CODE | [mV] | (+/−) | [mV] | (+/−) | [mV] | [mv] | (+/−) | ImV] |
| 1 | S9_5 | 19.5 | 1.35 | 13.57 | 0.61 | −5.93 | 20.5 | 0.62 | +6.93 |
| 2 | S9_6 | 41.93 | 1.58 | 25.9 | 1.56 | −16.03 | 24.37 | 1.12 | −1.53 |
| 3 | S10_4 | 43.4 | 3.03 | 28.83 | 5.52 | −14.57 | 20.6 | 0.96 | −8.23 |
| 4 | S10_5 | 42.1 | 2.86 | 38.27 | 0.67 | −3.83 | 34.57 | 5.42 | −3.7 |
| 5 | S10_6 | 51.9 | 2.26 | 32.9 | 1.08 | −19 | 25.93 | 1.12 | −6.97 |
| 6 | S11_4 | 47.43 | 1.12 | 36.4 | 1.82 | −11.03 | 30.87 | 1.76 | −5.53 |
| 7 | S11_5 | 48.87 | 1.76 | 35.63 | 1.27 | −13.23 | 29.43 | 0.9 | −6.2 |
| 8 | S11_6 | 49.63 | 0.67 | 32.33 | 1.56 | −17.3 | 29.57 | 1.67 | −2.77 |

The decrease in the value of the Zeta potential of the liposomes indicates RNA-m2 binding.

Example I is illustrated in FIG. 1A-F.

### Example II (Series II, S10_3 Liposome With Emetine and RNA-m2)

30 mg of emetine hydrochloride was dissolved in 1 mL of deionised water and then precipitated by titration with 0.2 M NaOH solution. The total volume of titrant utilised was 1.5 mL. The precipitate was centrifuged at 6000 RPM for 20 minutes and separated from the solution. 0.1 mL of 0.2 M NaOH was added to the supernatant solution to control complete precipitation of the emetine form. The amount of unprecipitated emetine was measured by UV-Vis spectroscopy against a calibration curve at pH 12.4. The precipitate was washed 5 times with distilled water, each time dispersing with ultrasound, centrifuging and measuring the amount of unprecipitated emetine in the supernatant solution. The total loss of emetine during the process was 3 mg (10%). The precipitate was dried in a vacuum dryer at 30° C., 100 mbar pressure for 5 h and used in the next synthetic steps. The solubility of the deprotonated form in water was found to be 1.79 mg/mL.

In order to prepare the S10_3 cationic liposomes, five times the weight of the lipids and emetine (according to Table 5) appropriate for series II were weighed, i.e. 0.475 mg DMPC 0.34 mg DPPC, 1.49 mg 16:0 EPC, 2.08 mg 14:0 EPC, 0.94 mg of DSPE-PEG-NH2, 1.13 mg of cholesterol and 0.03 mg of emetine, and then dissolved in 2.5 mL of a 2:1 volume ratio of chloroform and methanol. The lipid mixture was allowed to evaporate the solvents in a vacuum evaporator with the following parameters: T=35° C., p=200 mbar, t=4 h. A thin lipid bilayer film was formed during the evaporation process. The layer was dried in a vacuum dryer with the following parameters: T=35° C., p=200 mbar, t=1 h. After complete evaporation of the organic solvents, 22.5 mL of sterile saline filtered through bpPVDF, 0.2 μm syringe filters was added. The entirety was hydrated in a vacuum evaporator under the following conditions: T=50° C., p=300 mbar, t=6 h. Hydrated lipid layers were allowed to cool down and then sonicated at the temperature T=60° C., over the time of t=40 min. and at a frequency of 50 Hz. After this time, the colour of the solution changed from milky to colourless, which indicated the formation of small unilameller vesicles (SUVs). The solutions were then allowed to cool and the size of the liposomes was examined by the DLS dynamic light scattering method. The amount of bound emetine was monitored by the UV-Vis method at a wavelength of 280 nm. The obtained liposomes were purified by dialysis on 10 kD membranes with ultra pure water (Milli-Q system, Biopak CDUFBI001 filter: RNase free, DNase-free, Pyrogen-free) for 50 minutes (4.5 mL of sample, 1 L), changing the water three times after 20 min, 30 min and 40 min. The purity of the obtained liposomes was measured by UV-Vis spectroscopy. The Zeta potential and size were then measured using the DLS method and the composition characterization performed using the ATR IR method. The concentration of the obtained liposomes was 0.29 mg/mL.

The measurement results for the S10_3 liposomes and other series II liposomes are presented in Table 6.

0.250 μL of RNA-m2 solution (20 μg/mL) dissolved in ultra pure water (Milli-Q system, Biopak CDUFBI001 filter: RNase free, DNase-free, Pyrogen-free), additionally sterilised twice in a medical steriliser, was added to 0.75 mL of S11_3 liposomes from series II. Next, the entirety was tightly closed, shielded from light sources and incubated at room temperature for 1 hour. After the incubation period, the size and zeta potential of each system were measured to exclude liposome aggregation and confirm RNA-m2 binding (Zeta potential). Next, another 0.250 μL of RNA-m2 solution (20 μg/mL) was added, the entirety was incubated for another hour, and the zeta potential was measured to determine the RNA-m2 loading capacity of the liposomes. Additionally, measurements of the size of the systems were performed after 5 days to investigate the stability of the liposomes. The measurement results for the S10_3 system are presented in Tables 7 and 8. The Tables also include the results for all series II liposomes.

TABLE 5
Composition of series II liposomes expressed as molar, mass and percentage ratios.

| | | | | | | | | | | |
|---|---|---|---|---|---|---|---|---|---|---|
| | % by weight | | | | | | | | | |
| | of cationic | System | | | 16:0 | 14:0 | DSPE- | | DSPE- | |
| No. | phospholipids | CODE | DMPC | DPPC | EPC | EPC | PEG-NH2 | Cholesterol | PEG-FA | Emetine |

| | | | |
|---|---|---|---|
| 1 | 23.63 | S9_2 | Molar ratio |

| 1.2 | 0.16 | 0.64 | 0 | 0.116 | 1 | 0 | 0.0225 |
|---|---|---|---|---|---|---|---|
| Mass ratio [mg] | | | | | | | |
| 0.473 | 0.068 | 0.297 | 0 | 0.188 | 0.225 | 0 | 0.006 |
| Percentage by weight [%] | | | | | | | |
| 37.63 | 5.41 | 23.63 | 0.00 | 14.96 | 17.90 | 0.00 | 0.48 |

| | | | |
|---|---|---|---|
| 2 | 29.43 | S9_3 | Molar ratio |

| 1.2 | 0 | 0.8 | 0 | 0.116 | 1 | 0 | 0.0225 |
|---|---|---|---|---|---|---|---|
| Mass ratio [mg] | | | | | | | |
| 0.473 | 0 | 0.372 | 0 | 0.188 | 0.225 | 0 | 0.006 |
| Percentage by weight [%] | | | | | | | |
| 37.42 | 0.00 | 29.43 | 0.00 | 14.87 | 17.80 | 0.00 | 0.47 |

| | | | |
|---|---|---|---|
| 3 | 24.66 | S10_1 | Molar ratio |

| 0.48 | 0.8 | 0 | 0.72 | 0.116 | 1 | 0 | 0.0225 |
|---|---|---|---|---|---|---|---|
| Mass ratio [mg] | | | | | | | |
| 0.189 | 0.342 | 0 | 0.311 | 0.188 | 0.225 | 0 | 0.006 |
| Percentage by weight [%] | | | | | | | |
| 14.99 | 27.12 | 0.00 | 24.66 | 14.91 | 17.84 | 0.00 | 0.48 |

| | | | |
|---|---|---|---|
| 4 | 32.65 | S10_2 | Molar ratio |

| 0.24 | 0.8 | 0 | 0.96 | 0.116 | 1 | 0 | 0.0225 |
|---|---|---|---|---|---|---|---|
| Mass ratio [mg] | | | | | | | |
| 0.095 | 0.342 | 0 | 0.415 | 0.188 | 0.225 | 0 | 0.006 |
| Percentage by weight [%] | | | | | | | |
| 7.47 | 26.91 | 0.00 | 32.65 | 14.79 | 17.70 | 0.00 | 0.47 |

| | | | |
|---|---|---|---|
| 5 | 40.50 | S10_3 | Molar ratio |

| 0 | 0.8 | 0 | 1.2 | 0.116 | 1 | 0 | 0.0225 |
|---|---|---|---|---|---|---|---|
| Mass ratio [mg] | | | | | | | |
| 0 | 0.342 | 0 | 0.518 | 0.188 | 0.225 | 0 | 0.006 |
| Percentage by weight [%] | | | | | | | |
| 0.00 | 26.74 | 0.00 | 40.50 | 14.70 | 17.59 | 0.00 | 0.47 |

| | | | |
|---|---|---|---|
| 6 | 28.16 | S11_1 | Molar ratio |

| 0.72 | 0.48 | 0.32 | 0.48 | 0.116 | 1 | 0 | 0.0225 |
|---|---|---|---|---|---|---|---|
| Mass ratio [mg] | | | | | | | |
| 0.284 | 0.205 | 0.149 | 0.207 | 0.188 | 0.225 | 0 | 0.006 |
| Percentage by weight [%] | | | | | | | |
| 22.47 | 16.22 | 11.79 | 16.38 | 14.87 | 17.80 | 0.00 | 0.47 |

| | | | |
|---|---|---|---|
| 7 | 41.75 | S11_2 | Molar ratio |

| 0.48 | 0.32 | 0.48 | 0.72 | 0.116 | 1 | 0 | 0.0225 |
|---|---|---|---|---|---|---|---|
| Mass ratio [mg] | | | | | | | |
| 0.189 | 0.137 | 0.223 | 0.311 | 0.188 | 0.225 | 0 | 0.006 |
| Percentage by weight [%] | | | | | | | |
| 14.78 | 10.71 | 17.44 | 24.32 | 14.70 | 17.59 | 0.00 | 0.47 |

| | | | |
|---|---|---|---|
| 8 | 55.02 | S11_3 | Molar ratio |

| 0.24 | 0.16 | 0.64 | 0.96 | 0.116 | 1 | 0 | 0.0225 |
|---|---|---|---|---|---|---|---|
| Mass ratio [mg] | | | | | | | |
| 0.095 | 0.068 | 0.297 | 0.415 | 0.188 | 0.225 | 0 | 0.006 |
| Percentage by weight [%] | | | | | | | |
| 7.34 | 5.26 | 22.95 | 32.07 | 14.53 | 17.39 | 0.00 | 0.46 |

TABLE 6
Physical parameters of the series II liposomes
with standard deviations (SD).

| | | Physical parameters | | | | | |
|---|---|---|---|---|---|---|---|
| | | hydrodynamic | | polydis- | | | |
| | System | radius HR | SD | persity | SD | Zeta | SD |
| No. | CODE | [nm] | (+/−) | factor | (+/−) | [mV] | (+/−) |
| 1 | S9_2 | 81.32 | 1.79 | 0.151 | 0.025 | 25.9 | 3.7 |
| 2 | S9_3 | 80.5 | 2.9 | 0.164 | 0.019 | 23.07 | 0.31 |
| 3 | S10_1 | 67.74 | 2.27 | 0.152 | 0.003 | 34.3 | 11.75 |
| 4 | S10_2 | 101.97 | 0.29 | 0.112 | 0.012 | 49.45 | 0.07 |
| 5 | S10_3 | 96.04 | 0.74 | 0.063 | 0.005 | 40.73 | 1.42 |
| 6 | S11_1 | 81.54 | 1.11 | 0.108 | 0.013 | 49.57 | 2.17 |
| 7 | S11_2 | 110.63 | 0.55 | 0.09 | 0.014 | 45.57 | 2 |
| 8 | S11_3 | 125.6 | 3.32 | 0.073 | 0.02 | 50.1 | 2.09 |

TABLE 7
Size (hydrodynamic radius, HR) and polydispersity index (PDI) of liposomes
before and after the addition of 5 μg RNA-m2 per 1 mg of liposomes,
after one hour and five days with standard deviations (SD)

| | | | | | | After addition of | | | | 5 days after the addition | | | |
|---|---|---|---|---|---|---|---|---|---|---|---|---|---|
| | | Before the addition | | | | RNA-m2 5 μg per | | | | of RNA-m2 5 μg per | | | |
| | | of RNA-m2 | | | | 1 mg of liposomes | | | | 1 mg of liposomes | | | |
| | System | HR | SD | | SD | HR | SD | | SD | HR | SD | | SD |
| No | CODE | [nm] | (+/−) | PDI | (+/−) | [nm] | (+/−) | PDI | (+/−) | [nm] | (+/−) | PDI | (+/−) |
| 1 | S9_2 | 81.3 | 1.8 | 0.151 | 0.025 | 85.9 | 1 | 0.119 | 0.008 | 85.7 | 1.8 | 0.098 | 0.004 |
| 2 | S9_3 | 80.5 | 2.9 | 0.164 | 0.019 | 77.8 | 2.6 | 0.17 | 0.006 | 78 | 0.8 | 0.163 | 0.006 |
| 3 | S10_1 | 67.7 | 2.3 | 0.152 | 0.003 | 76 | 0.8 | 0.125 | 0.01 | 77.3 | 0.4 | 0.126 | 0.007 |
| 4 | S10_2 | 102 | 0.3 | 0.112 | 0.012 | 102.6 | 2.3 | 0.091 | 0.012 | 101.2 | 1 | 0.083 | 0.003 |
| 5 | S10_3 | 96 | 0.7 | 0.063 | 0.005 | 105.5 | 1.4 | 0.08 | 0.016 | 107.1 | 0.5 | 0.076 | 0.004 |
| 6 | S11_1 | 81.5 | 1.1 | 0.108 | 0.013 | 91 | 0.8 | 0.104 | 0.015 | 90.7 | 1 | 0.074 | 0.013 |
| 7 | S11_2 | 110.6 | 0.6 | 0.09 | 0.014 | 129.6 | 0.9 | 0.107 | 0.005 | 126.3 | 0.8 | 0.086 | 0.003 |
| 8 | S11_3 | 125.6 | 3.3 | 0.073 | 0.02 | 144.1 | 0.8 | 0.071 | 0.007 | 143.2 | 2.8 | 0.075 | 0.027 |

The lack of significant changes in both the size and the polydispersity coefficient prove the lack of liposome aggregation, i.e. their stability in the presence of RNA-m2.

TABLE 10
Changes in the Zeta potential of liposomes after the addition of 5 μg and 10 μg RNA-m2.

| | | | | After addition of 5 μg RNA- | | | After addition of 10 μg RNA- | | |
|---|---|---|---|---|---|---|---|---|---|
| | | | | m2 per 1 mg of liposomes | | | m2 per 1 mg of liposomes | | |
| | | Before the addition | | | | Zeta | | | Zeta |
| | | of RNA m2 | | | | potential | | | potential |
| | System | Zeta | SD | Zeta | SD | change | Zeta | SD | change |
| Liposome | CODE | [mv] | (+/−) | [mV] | (+/−) | [mV] | [mV] | (+/−) | [mV] |
| 1 | S9_2 | 25.9 | 3.7 | 12.83 | 1.04 | −13.07 | 24.57 | 1.7 | +11.73 |
| 2 | S9_3 | 23.07 | 0.31 | 11.4 | 1.08 | −11.67 | 20.47 | 0.9 | −9.07 |
| 3 | S10_1 | 34.3 | 11.75 | 22.55 | 2.05 | −11.75 | 22.35 | 0.78 | −0.2 |
| 4 | S10_2 | 49.45 | 0.07 | 38.53 | 0.9 | −10.92 | 27.23 | 1.1 | −11.3 |
| 5 | S10_3 | 40.73 | 1.42 | 37.43 | 2.27 | −3.3 | 36.17 | 0.35 | −1.27 |
| 6 | S11_1 | 49.57 | 2.17 | 30.63 | 3.91 | −18.93 | 30.53 | 2.05 | −0.1 |
| 7 | S11_2 | 45.57 | 2 | 34.97 | 2.53 | −10.6 | 26.9 | 1.49 | −8.07 |
| 8 | S11_3 | 50.1 | 2.09 | 36.1 | 2 | −14 | 31.03 | 1.16 | −5.07 |
| 9 | S11_6 | 49.63 | 0.67 | 32.33 | 1.56 | −17.3 | 29.57 | 1.67 | −2.77 |

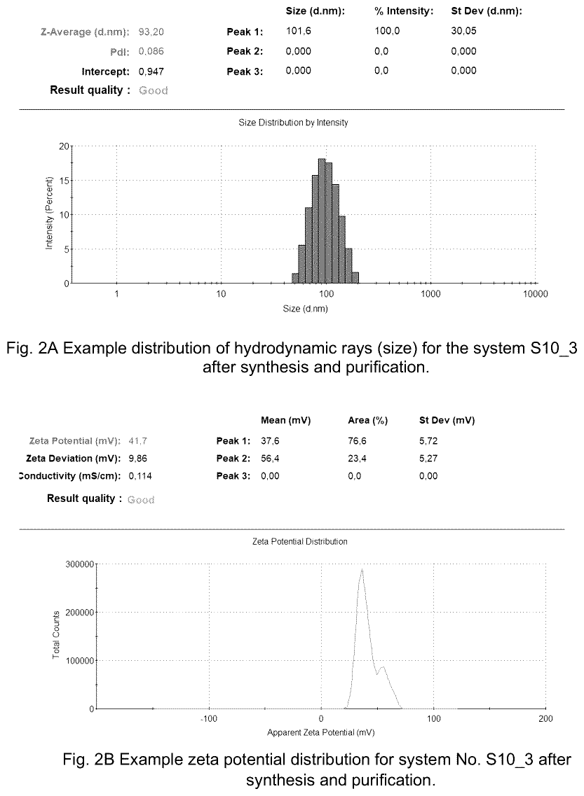

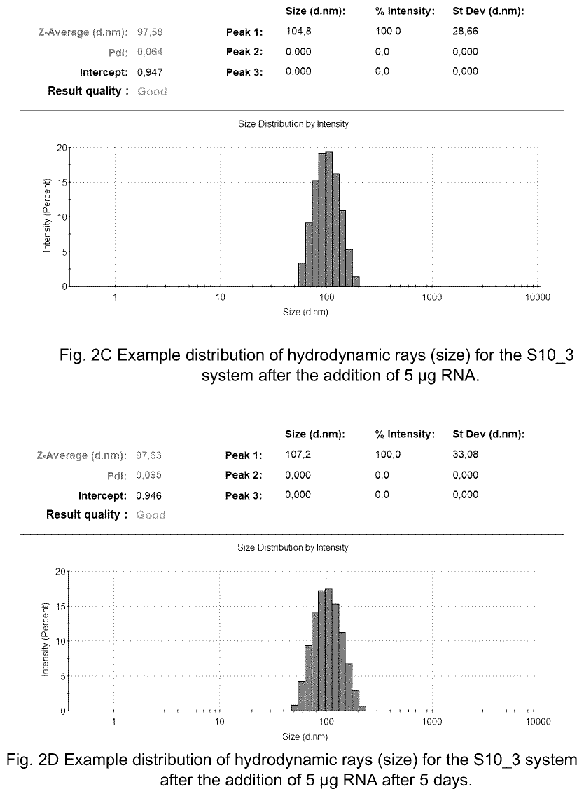

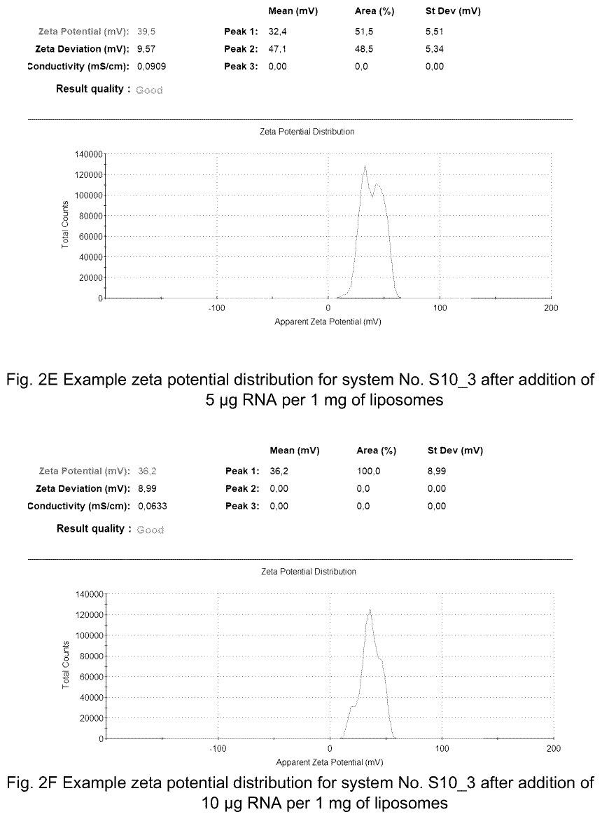

Example II is illustrated in FIG. 2A-F

### Example III (Series III, S18_2 Liposomes With RNA-m2, Emetine and Folic Acid Targeting Agent—FA)

30 mg of emetine hydrochloride was dissolved in 1 mL of deionised water and then precipitated by titration with 0.2 M NaOH solution. The total volume of titrant utilised was 1.5 mL. The precipitate was centrifuged at 6000 RPM for 20 minutes and separated from the solution. 0.1 mL of 0.2M NaOH was added to the supernatant solution to control complete precipitation of the emetine form. The amount of unprecipitated emetine was measured by UV-Vis spectroscopy against a calibration curve at pH 12.4. The precipitate was washed 5 times with distilled water, each time dispersing with ultrasound, centrifuging and measuring the amount of unprecipitated emetine in the supernatant solution. The total loss of emetine during the process was 3 mg (10%). The precipitate was dried in a vacuum dryer at 30° C., 100 mbar pressure for 5 h and used in the next synthetic steps. The solubility of the deprotonated form in water was found to be 1.79 mg/mL.

In order to prepare the S18_2 cationic liposomes, five times the weight of the lipids and emetine (according to Table 11) appropriate for series III were weighed, i.e. 0.48 mg DMPC, 2.38 mg DPPC, 2.08 14:0 EPC, 0.94 mg DSPE-PEG-NH2, 1.13 mg of cholesterol, 0.095 mg of DSPE-PEG-FA and 0.03 mg of emetine, and then dissolved in 2.5 mL of a 2:1 volume ratio of chloroform and methanol. The lipid mixture was allowed to evaporate the solvents in a vacuum evaporator with the following parameters: T=35° C., p=200 mbar, t=4 h. A thin lipid bilayer film was formed during the evaporation process. The layer was dried in a vacuum dryer with the following parameters: T=40° C., p=200 mbar, t=1 h. After complete evaporation of the organic solvents, 22.5 mL of sterile saline filtered through bpPVDF, 0.2 μm syringe filters was added. The entirety was hydrated in a vacuum evaporator under the following conditions: T=50° C., p=300 mbar, t=6 h. Hydrated lipid layers were allowed to cool down and then sonicated at the temperature T=60° C., over the time of t=40 min. and at a frequency of 50 Hz. After this time, the colour of the solution changed from milky to colourless, which indicated the formation of small unilameller vesicles (SUVs). The solutions were then allowed to cool and the size of the liposomes was examined by the DLS dynamic light scattering method. The amount of bound emetine was monitored by the UV-Vis method at a wavelength of 280 nm. The obtained liposomes were purified by dialysis on 10 kD membranes with ultra pure water (Milli-Q system, Biopak CDUFBI001 filter: RNase free, DNase-free, Pyrogen-free) for 50 minutes (4.5 mL of sample, 1 L), changing the water three times after 20 min, 30 min and 40 min. The purity of the obtained liposomes was measured by UV-Vis spectroscopy. The Zeta potential and size were then measured using the DLS method and the composition characterization performed using the ATR IR method. The concentration of the obtained liposomes was 0.28 mg/mL. The results of measurements for the S18_2 liposomes and other series III liposomes are presented in Table 12.

TABLE 11
Composition of series III liposomes expressed as molar, mass and percentage ratios.

| | | | | | | | | | | |
|---|---|---|---|---|---|---|---|---|---|---|
| | % by weight | | | | | | | | | |
| | of cationic | System | | | 16:0 | 14:0 | DSPE- | | DSPE- | |
| Liposomes | phospholipids | CODE | DMPC | DPPC | EPC | EPC | PEG-NH2 | Cholesterol | PEG-FA | Emetine |

| | | | |
|---|---|---|---|
| 1 | 23.28 | S17_2 | Molar ratio |

| 1.2 | 0.16 | 0.64 | 0 | 0.116 | 1 | 0.01 | 0.0225 |
|---|---|---|---|---|---|---|---|
| Mass ratio [mg] | | | | | | | |
| 0.473 | 0.068 | 0.297 | 0 | 0.188 | 0.225 | 0.019 | 0.006 |
| Percentage by weight [%] | | | | | | | |
| 37.63 | 5.41 | 23.63 | 0.00 | 14.96 | 17.90 | 1.51 | 0.48 |

| | | | |
|---|---|---|---|
| 2 | 28.99 | S17_3 | Molar ratio |

| 1.2 | 0 | 0.8 | 0 | 0.116 | 1 | 0.01 | 0.0225 |
|---|---|---|---|---|---|---|---|
| Mass ratio [mg] | | | | | | | |
| 0.473 | 0 | 0.372 | 0 | 0.188 | 0.225 | 0.019 | 0.006 |
| Percentage by weight [%] | | | | | | | |
| 37.42 | 0.00 | 29.43 | 0.00 | 14.87 | 17.80 | 1.50 | 0.47 |

| | | | |
|---|---|---|---|
| 3 | 24.30 | S18_1 | Molar ratio |

| 0.48 | 0.8 | 0 | 0.72 | 0.116 | 1 | 0.01 | 0.0225 |
|---|---|---|---|---|---|---|---|
| Mass ratio [mg] | | | | | | | |
| 0.189 | 0.342 | 0 | 0.311 | 0.188 | 0.225 | 0.019 | 0.006 |
| Percentage by weight [%] | | | | | | | |
| 14.99 | 27.12 | 0.00 | 24.66 | 14.91 | 17.84 | 1.51 | 0.48 |

| | | | |
|---|---|---|---|
| 4 | 32.17 | S18_2 | Molar ratio |

| 0.24 | 0.8 | 0 | 0.96 | 0.116 | 1 | 0.01 | 0.0225 |
|---|---|---|---|---|---|---|---|
| Mass ratio [mg] | | | | | | | |
| 0.095 | 0.342 | 0 | 0.415 | 0.188 | 0.225 | 0.019 | 0.006 |
| Percentage by weight [%] | | | | | | | |
| 7.47 | 26.91 | 0.00 | 32.65 | 14.79 | 17.70 | 1.49 | 0.47 |

| | | | |
|---|---|---|---|
| 5 | 39.91 | S18_3 | Molar ratio |

| 0 | 0.8 | 0 | 1.2 | 0.116 | 1 | 0.01 | 0.0225 |
|---|---|---|---|---|---|---|---|
| Mass ratio [mg] | | | | | | | |
| 0 | 0.342 | 0 | 0.518 | 0.188 | 0.225 | 0.019 | 0.006 |
| Percentage by weight [%] | | | | | | | |
| 0.00 | 26.74 | 0.00 | 40.50 | 14.70 | 17.59 | 1.49 | 0.47 |

| | | | |
|---|---|---|---|
| 6 | 27.75 | S19_1 | Molar ratio |

| 0.72 | 0.48 | 0.32 | 0.48 | 0.116 | 1 | 0.01 | 0.0225 |
|---|---|---|---|---|---|---|---|
| Mass ratio [mg] | | | | | | | |
| 0.284 | 0.205 | 0.149 | 0.207 | 0.188 | 0.225 | 0.019 | 0.006 |
| Percentage by weight [%] | | | | | | | |
| 22.47 | 16.22 | 11.79 | 16.38 | 14.87 | 17.80 | 1.50 | 0.47 |

| | | | |
|---|---|---|---|
| 7 | 41.14 | S19_2 | Molar ratio |

| 0.48 | 0.32 | 0.48 | 0.72 | 0.116 | 1 | 0.01 | 0.0225 |
|---|---|---|---|---|---|---|---|
| Mass ratio [mg] | | | | | | | |
| 0.189 | 0.137 | 0.223 | 0.311 | 0.188 | 0.225 | 0.019 | 0.006 |
| Percentage by weight [%] | | | | | | | |
| 14.78 | 10.71 | 17.44 | 24.32 | 14.70 | 17.59 | 1.49 | 0.47 |

| | | | |
|---|---|---|---|
| 8 | 54.23 | S19_3 | Molar ratio |

| 0.24 | 0.16 | 0.64 | 0.96 | 0.116 | 1 | 0.01 | 0.0225 |
|---|---|---|---|---|---|---|---|
| Mass ratio [mg] | | | | | | | |
| 0.095 | 0.068 | 0.297 | 0.415 | 0.188 | 0.225 | 0.019 | 0.006 |
| Percentage by weight [%] | | | | | | | |
| 7.34 | 5.26 | 22.95 | 32.07 | 14.53 | 17.39 | 1.47 | 0.46 |

TABLE 12
Physical parameters of the series III liposomes
with standard deviations (SD).

| | | Physical parameters | | | | | |
|---|---|---|---|---|---|---|---|
| | | hydro- | | PDI | | | |
| | | dynamic | | polydis- | | Zeta | |
| | System | radius | SD | persity | SD | potential | SD |
| No. | CODE | [nm] | (+/−) | index | (+/−) | [mV] | (+/−) |
| 1 | S17_2 | 154.83 | 0.91 | 0.158 | 0.013 | 28.13 | 1.8 |
| 2 | S17_3 | 93.69 | 1.48 | 0.107 | 0.009 | 34.9 | 0.62 |
| 3 | S18_1 | 83.31 | 1.2 | 0.134 | 0.01 | 37 | 0.75 |
| 4 | S18_2 | 97.55 | 0.85 | 0.08 | 0.019 | 44.1 | 1.68 |
| 5 | S18_3 | 84.57 | 1.36 | 0.093 | 0.02 | 54.93 | 2.11 |
| 6 | S19_1 | 86.19 | 5.38 | 0.16 | 0.019 | 39.4 | 0.85 |
| 7 | S19_2 | 88.34 | 5.13 | 0.157 | 0.006 | 39.9 | 3.63 |
| 8 | S19_3 | 103.03 | 0.98 | 0.118 | 0.025 | 50.3 | 4.54 |

0.250 μL of RNA-m2 solution (20 μg/mL) dissolved in ultra pure water (Milli-Q system, Biopak CDUFBI001 filter: RNase free, DNase-free, Pyrogen-free), additionally sterilised twice in a medical steriliser, was added to 0.75 mL of S18_2 liposomes from series III. Next, the entirety was tightly closed, shielded from light sources and incubated at room temperature for 1 hour. After the incubation period, the size and zeta potential of each system were measured to exclude liposome aggregation and confirm RNA-m2 binding (Zeta potential). Next, another 0.250 μL of RNA-m2 solution (20 μg/mL) was added, the entirety was incubated for another hour, and the zeta potential was measured to determine the RNA-m2 loading capacity of the liposomes. Additionally, measurements of the size of the systems were performed after 5 days to investigate the stability of the liposomes. The measurement results for the S18_2 system are presented in Tables 13 and 14. The Tables also include the results for all series III liposomes.

TABLE 13
Size (hydrodynamic radius, HR) and polydispersity index (PDI) of liposomes
from series III before and after the addition of 5 μg RNA-m2 per 1 mg
of liposomes, after one hour and five days with standard deviations (SD)

| | | | | | | After addition of | | | | 5 days after the addition | | | |
|---|---|---|---|---|---|---|---|---|---|---|---|---|---|
| | | | | | | RNA-m2 5 μg per | | | | of RNA-m2 5 μg per | | | |
| | | Before RNA addition | | | | 1 mg of liposomes | | | | 1 mg of liposomes | | | |
| | System | HR | SD | | SD | HR | SD | | SD | HR | SD | | SD |
| Liposome | CODE | [nm] | (+/−) | PDI | (+/−) | [nm] | (+/−) | PDI | (+/−) | [nm] | (+/−) | PDI | (+/−) |
| 1 | S17_2 | 154.8 | 0.9 | 0.158 | 0.013 | 148.6 | 1.4 | 0.205 | 0.012 | 191.8 | 4 | 0.205 | 0.008 |
| 2 | S17_3 | 93.7 | 1.5 | 0.107 | 0.009 | 94.5 | 0.1 | 0.082 | 0.017 | 102.5 | 1.4 | 0.089 | 0.014 |
| 3 | S18_1 | 83.3 | 1.2 | 0.134 | 0.01 | 90.6 | 0.5 | 0.126 | 0.004 | 101.2 | 1 | 0.115 | 0.007 |
| 4 | S18_2 | 97.6 | 0.8 | 0.08 | 0.019 | 87.8 | 0.5 | 0.13 | 0.011 | 97.7 | 2.3 | 0.121 | 0.005 |
| 5 | S18_3 | 84.6 | 1.4 | 0.093 | 0.02 | 91 | 0.9 | 0.095 | 0.006 | 96 | 3.1 | 0.104 | 0.02 |
| 6 | S19_1 | 86.2 | 5.4 | 0.16 | 0.019 | 94 | 0.7 | 0.099 | 0.011 | 102.8 | 1.1 | 0.08 | 0.011 |
| 7 | S19_2 | 88.3 | 5.1 | 0.157 | 0.006 | 90 | 0.3 | 0.086 | 0.007 | 100.8 | 0.4 | 0.088 | 0.016 |
| 8 | S19_3 | 103 | 1 | 0.118 | 0.025 | 106.9 | 0.3 | 0.079 | 0.027 | 116.6 | 0.8 | 0.066 | 0.01 |

The lack of significant changes in both the size and the polydispersity coefficient prove the lack of liposome aggregation, i.e. their stability in the presence of RNA-m2.

TABLE 14
Changes in the Zeta potential of series III liposomes after the addition
of 5 μg and 10 μg RNA-m2 along with standard deviations (SD).

| | | | | After addition of 5 μg RNA- | | | After addition of 10 μg RNA- | | |
|---|---|---|---|---|---|---|---|---|---|
| | | Before the addition | | m2 per 1 mg of liposomes | | | m2 per 1 mg of liposomes | | |
| | | of RNA-m2 | | | | Zeta | | | Zeta |
| | | Zeta | | Zeta | | potential | | | potential |
| | System | potential | SD | potential | SD | change | Zeta | SD | change |
| No. | CODE | [mv] | (+/−) | [mv] | (+/−) | [mV] | [mv] | (+/−) | [mV] |
| 1 | S17_2 | 28.13 | 1.8 | 24.07 | 0.47 | −4.07 | 21.37 | 2.25 | −2.7 |
| 2 | S17_3 | 34.9 | 0.62 | 21.77 | 0.91 | −13.13 | 20.37 | 2.97 | −1.4 |
| 3 | S18_1 | 37 | 0.75 | 28.77 | 1.12 | −8.23 | 24.07 | 0.61 | −4.7 |
| 4 | S18_2 | 44.1 | 1.68 | 37.83 | 0.55 | −6.27 | 30.83 | 2.17 | −7 |
| 5 | S18_3 | 54.93 | 2.11 | 42.7 | 0.82 | −12.23 | 30.1 | 1.54 | −12.6 |
| 6 | S19_1 | 39.4 | 0.85 | 35.73 | 1.27 | −3.67 | 25.3 | 2.97 | −10.43 |
| 7 | S19_2 | 39.9 | 3.63 | 35.6 | 0.8 | −4.3 | 27.97 | 2.06 | −7.63 |
| 8 | S19_3 | 50.3 | 4.54 | 47.57 | 0.49 | −2.73 | 35 | 0.62 | −12.57 |

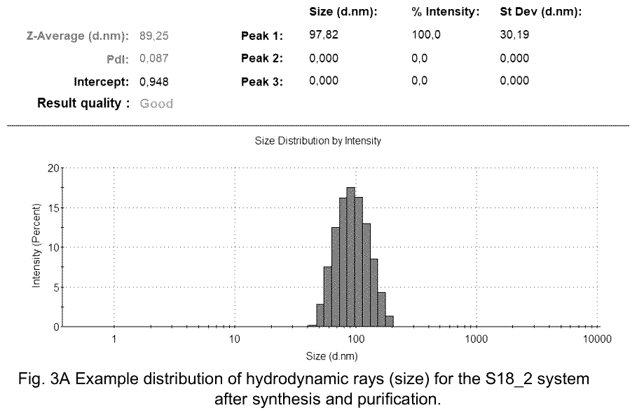

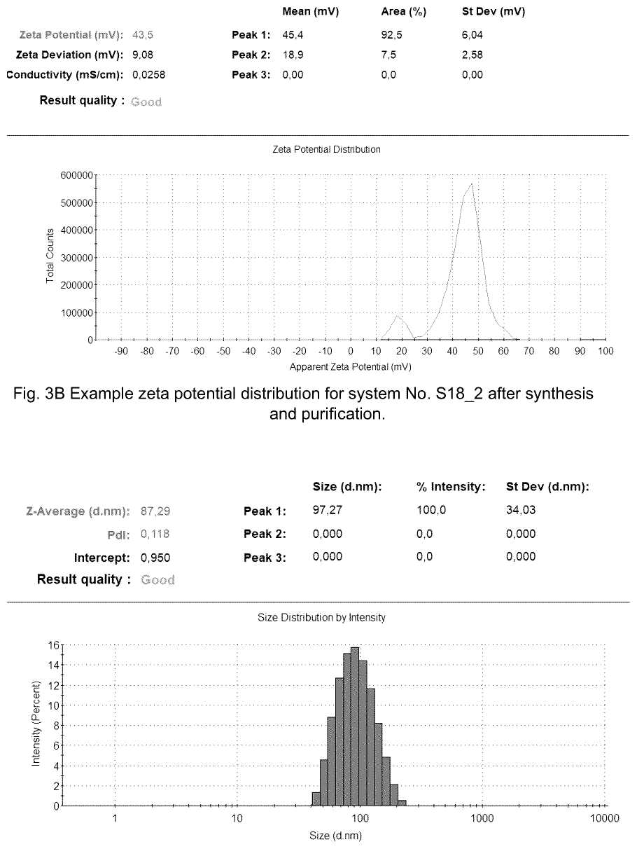

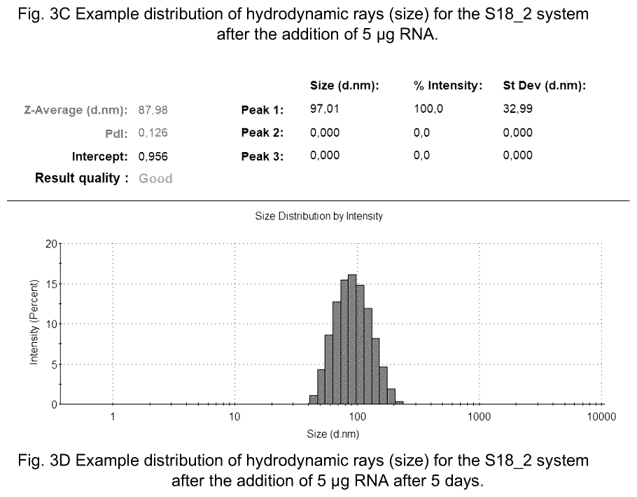

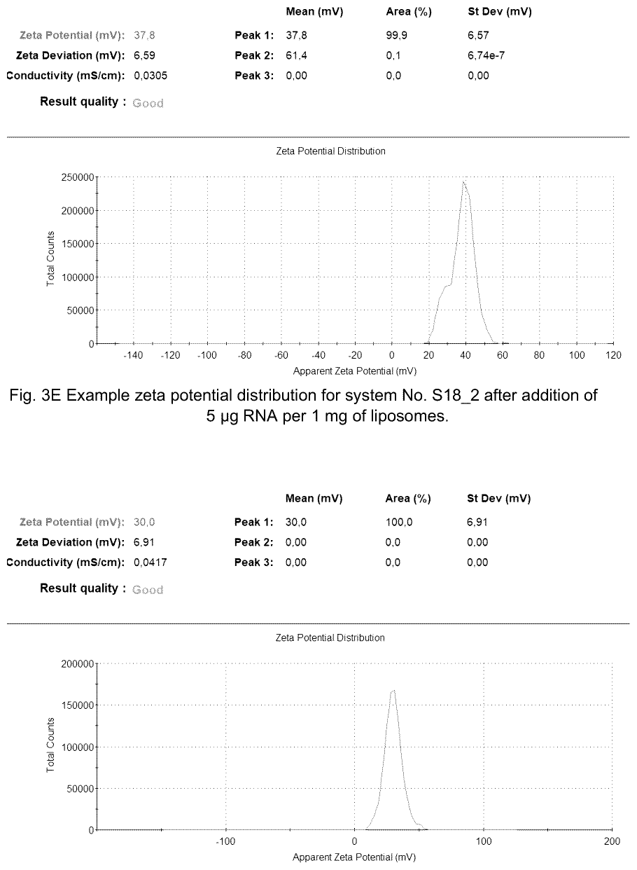

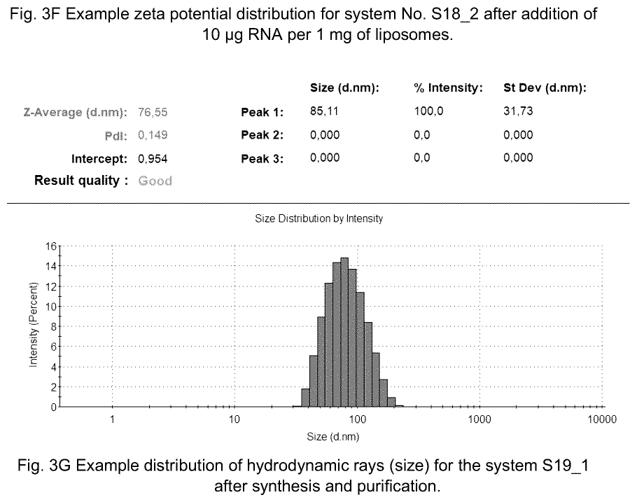

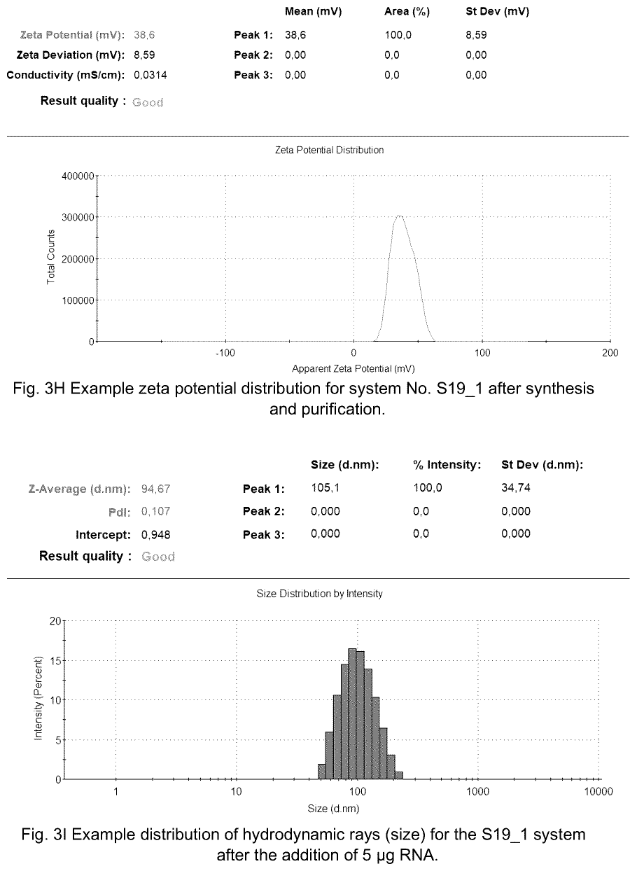

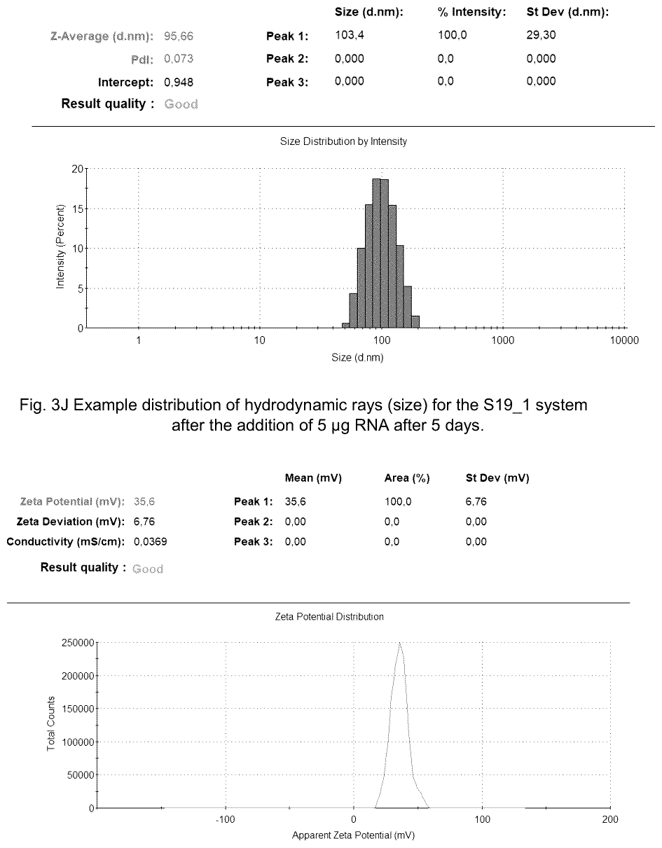

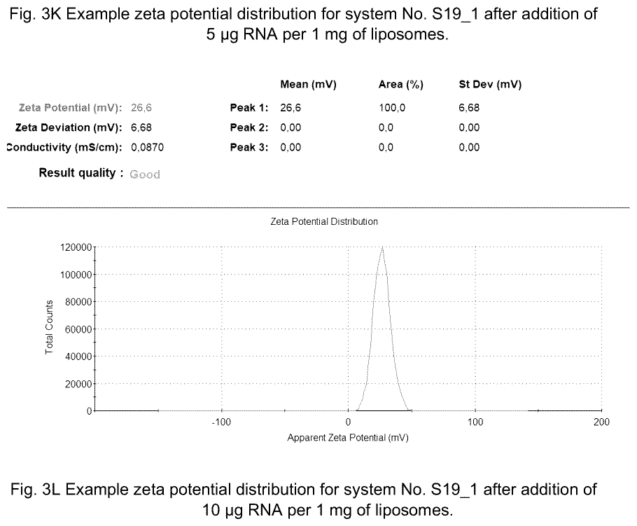

Example III is illustrated in FIG. 3A-L

### Example IV (Series IV, S17_4 Liposome With RNA and Folic Acid Targeting Factor—FA, Without Emetine)

In order to prepare cationic liposomes S17_2, the appropriate lipid masses were weighed according to Table 15, i.e. 0.473 mg DMPC, 0.137 mg DPPC, 0.233 mg 16:0 EPC, 0.188 mg DSPE-PEG-NH2, 0.225 mg cholesterol and 0.019 mg DSPE-PEG-NH2 and then dissolved in 0.5 mL of a 2:1 volume ratio mixture of chloroform with methanol. The lipid mixture was allowed to evaporate the solvents during t=3 h in a vacuum dryer at T=25° C., pressure p=100 mbar. A thin lipid bilayer film was formed during the evaporation process. After complete evaporation of the organic solvents, 4.5 mL of sterile saline filtered through bpPVDF, 0.2 μm syringe filters was added. The entirety was tightly closed and secured with parafilm, and then hydrated in a vacuum dryer: T=50° C., p=200 mbar, t=10 h. Hydrated lipid layers were allowed to cool down and then sonicated in accordance with the following parameters: T=65° C., t=30 min., 50 Hz. After this time, the colour of the solution changed from milky to colourless, which indicated the formation of small unilameller vesicles (SUVs). The solutions were then allowed to cool and the size of the liposomes was examined by the DLS dynamic light scattering method. The obtained liposomes were purified by dialysis on 10 kD membranes with ultra pure water (Milli-Q system, Biopak CDUFBI001 filter: RNase free, DNase-free, Pyrogen-free) for 40 minutes (4.5 mL of sample per 1 L of water), changing the water three times with changes after 10 minutes, 20 minutes and 30 minutes. The purity of the obtained liposomes was measured by UV-Vis spectroscopy. Zeta potential and size were then measured using the DLS method, and composition characterization was performed using the ATR IR method. The measurement results are presented in Table 16.

TABLE 15
Composition of series IV liposomes expressed as molar, mass and percentage ratios.

| | | | | | | | | | | |
|---|---|---|---|---|---|---|---|---|---|---|
| | % by weight | | | | | | | | | |
| | of cationic | System | | | 16:0 | 14:0 | DSPE- | | DSPE- | |
| No. | phospholipids | CODE | DMPC | DPPC | EPC | EPC | PEG-NH2 | Cholesterol | PEG-FA | Emetine |

| | | | |
|---|---|---|---|
| 1 | 17.63 | S17_4 | Molar ratio |

| 1.2 | 0.32 | 0.48 | 0 | 0.116 | 1 | 0.01 | 0 |
|---|---|---|---|---|---|---|---|
| Mass ratio [mg] | | | | | | | |
| 0.473 | 0.137 | 0.223 | 0 | 0.188 | 0.225 | 0.019 | 0 |
| Percentage by weight [%] | | | | | | | |
| 37.96 | 11.00 | 17.90 | 0.00 | 15.09 | 18.06 | 1.52 | 0.00 |

| | | | |
|---|---|---|---|
| 2 | 23.39 | S17_5 | Molar ratio |

| 1.2 | 0.16 | 0.64 | 0 | 0.116 | 1 | 0.01 | 0 |
|---|---|---|---|---|---|---|---|
| Mass ratio [mg] | | | | | | | |
| 0.473 | 0.068 | 0.297 | 0 | 0.188 | 0.225 | 0.019 | 0 |
| Percentage by weight [%] | | | | | | | |
| 37.81 | 5.44 | 23.74 | 0.00 | 15.03 | 17.99 | 1.52 | 0.00 |

| | | | |
|---|---|---|---|
| 3 | 29.13 | S17_6 | Molar ratio |

| 1.2 | 0 | 0.8 | 0 | 0.116 | 1 | 0.01 | 0 |
|---|---|---|---|---|---|---|---|
| Mass ratio [mg] | | | | | | | |
| 0.473 | 0 | 0.372 | 0 | 0.188 | 0.225 | 0.019 | 0 |
| Percentage by weight [%] | | | | | | | |
| 37.60 | 0.00 | 29.57 | 0.00 | 14.94 | 17.89 | 1.51 | 0.00 |

| | | | |
|---|---|---|---|
| 4 | 24.41 | S18_4 | Molar ratio |

| 0.48 | 0.8 | 0 | 0.72 | 0.116 | 1 | 0.01 | 0 |
|---|---|---|---|---|---|---|---|
| Mass ratio [mg] | | | | | | | |
| 0.189 | 0.342 | 0 | 0.311 | 0.188 | 0.225 | 0.019 | 0 |
| Percentage by weight [%] | | | | | | | |
| 15.06 | 27.25 | 0.00 | 24.78 | 14.98 | 17.93 | 1.51 | 0.00 |

| | | | |
|---|---|---|---|
| 5 | 32.32 | S18_5 | Molar ratio |

| 0.24 | 0.8 | 0 | 0.96 | 0.116 | 1 | 0.01 | 0 |
|---|---|---|---|---|---|---|---|
| Mass ratio [mg] | | | | | | | |
| 0.095 | 0.342 | 0 | 0.415 | 0.188 | 0.225 | 0.019 | 0 |
| Percentage by weight [%] | | | | | | | |
| 7.51 | 27.04 | 0.00 | 32.81 | 14.86 | 17.79 | 1.50 | 0.00 |

| | | | |
|---|---|---|---|
| 6 | 40.09 | S18_6 | Molar ratio |

| 0 | 0.8 | 0 | 1.2 | 0.116 | 1 | 0.01 | 0 |
|---|---|---|---|---|---|---|---|
| Mass ratio [mg] | | | | | | | |
| 0 | 0.342 | 0 | 0.518 | 0.188 | 0.225 | 0.019 | 0 |
| Percentage by weight [%] | | | | | | | |
| 0.00 | 26.87 | 0.00 | 40.69 | 14.77 | 17.67 | 1.49 | 0.00 |

| | | | |
|---|---|---|---|
| 7 | 27.88 | S19_4 | Molar ratio |

| 0.72 | 0.48 | 0.32 | 0.48 | 0.116 | 1 | 0.01 | 0 |
|---|---|---|---|---|---|---|---|
| Mass ratio [mg] | | | | | | | |
| 0.284 | 0.205 | 0.149 | 0.207 | 0.188 | 0.225 | 0.019 | 0 |
| Percentage by weight [%] | | | | | | | |
| 22.58 | 16.30 | 11.84 | 16.45 | 14.94 | 17.89 | 1.51 | 0.00 |

| | | | |
|---|---|---|---|
| 8 | 41.33 | S19_5 | Molar ratio |

| 0.48 | 0.32 | 0.48 | 0.72 | 0.116 | 1 | 0.01 | 0 |
|---|---|---|---|---|---|---|---|
| Mass ratio [mg] | | | | | | | |
| 0.189 | 0.137 | 0.223 | 0.311 | 0.188 | 0.225 | 0.019 | 0 |
| Percentage by weight [%] | | | | | | | |
| 14.85 | 10.76 | 17.52 | 24.43 | 14.77 | 17.67 | 1.49 | 0.00 |

| | | | |
|---|---|---|---|
| 9 | 54.48 | S19_6 | Molar ratio |

| 0.24 | 0.16 | 0.64 | 0.96 | 0.116 | 1 | 0.01 | 0 |
|---|---|---|---|---|---|---|---|
| Mass ratio [mg] | | | | | | | |
| 0.095 | 0.068 | 0.297 | 0.415 | 0.188 | 0.225 | 0.019 | 0 |
| Percentage by weight [%] | | | | | | | |
| 7.38 | 5.28 | 23.06 | 32.22 | 14.60 | 17.47 | 1.48 | 0.00 |

TABLE 16
Physical parameters of the series IV liposomes
with standard deviations (SD).

| | | Physical parameters | | | | | |
|---|---|---|---|---|---|---|---|
| | | Hydro- | | Polydis- | | | |
| | | dynamic | | persity | | Zeta | |
| | System | radius | SD | index | SD | potential | SD |
| No. | CODE | [nm] | (+/−) | PDI | (+/−) | [mv] | (+/−) |
| 1 | S17_4 | 82.8 | 2.34 | 0.173 | 0.008 | 36.67 | 1.24 |
| 2 | S17_5 | 86.14 | 0.21 | 0.14 | 0.009 | 36.93 | 4.89 |
| 3 | S17_6 | 105.64 | 8.03 | 0.212 | 0.026 | 47.97 | 0.58 |
| 4 | S18_4 | 157.27 | 4.41 | 0.159 | 0.027 | 47.67 | 1.31 |
| 5 | S18_5 | 99.73 | 0.77 | 0.107 | 0.01 | 49.27 | 1.45 |
| 6 | S18_6 | 79.54 | 5.22 | 0.17 | 0.017 | 50.9 | 1.86 |
| 7 | S19_4 | 103.03 | 0.98 | 0.118 | 0.025 | 46.4 | 2.23 |
| 8 | S19_5 | 97.8 | 4.17 | 0.163 | 0.013 | 50.73 | 3.63 |
| 9 | S19_6 | 124.33 | 2.93 | 0.137 | 0.008 | 56.07 | 2.16 |

0.250 μL of RNA-m2 solution (20 μg/mL) dissolved in ultra pure water (Milli-Q system, Biopak CDUFBI001 filter: RNase free, DNase-free, Pyrogen-free), additionally sterilised twice in a medical steriliser, was added to 0.75 mL of S17_4 liposomes from series IV. Next, the entirety was tightly closed, shielded from light sources and incubated at room temperature for 1 hour. After the incubation period, the size and zeta potential of each system were measured to exclude liposome aggregation and confirm RNA-m2 binding (Zeta potential). Next, another 0.250 μL of RNA-m2 solution (20 μg/mL) was added, the entirety was incubated for another hour, and the zeta potential was measured to determine the RNA-m2 loading capacity of the liposomes. Additionally, measurements of the size of the systems were performed after 5 days to investigate the stability of the liposomes. The measurement results for the S17_4 system are presented in Tables 17 and 18. The Tables also include the results for all series IV liposomes.

TABLE 17
Size (hydrodynamic radius, HR) and polydispersity index (PDI) of liposomes
from series IV before and after the addition of 5 μg RNA-m2 per 1 mg
of liposomes, after one hour and five days with standard deviations (SD)

| | | | | | | After addition of | | | | 5 days after the addition | | | |
|---|---|---|---|---|---|---|---|---|---|---|---|---|---|
| | | Before the addition | | | | RNA-m2 5 μg per | | | | of RNA-m2 5 μg per | | | |
| | | of RNA-m2 | | | | 1 mg of liposomes | | | | 1 mg of liposomes | | | |
| | System | HR | SD | | SD | HR | SD | | SD | HR | SD | | SD |
| No | CODE | [nm] | (+/−) | PDI | (+/−) | [nm] | (+/−) | PDI | (+/−) | [nm] | (+/−) | PDI | (+/−) |
| 1 | S17_4 | 82.8 | 2.3 | 0.173 | 0.008 | 77.7 | 0.7 | 0.159 | 0.007 | 90.1 | 2.7 | 0.151 | 0.01 |
| 2 | S17_5 | 86.1 | 0.2 | 0.14 | 0.009 | 88.5 | 0.8 | 0.109 | 0.015 | 98.4 | 1.9 | 0.099 | 0.013 |
| 3 | S17_6 | 105.6 | 8 | 0.212 | 0.026 | 110 | 1 | 0.124 | 0.007 | 127.5 | 1.7 | 0.13 | 0.006 |
| 4 | S18_4 | 157.3 | 4.4 | 0.159 | 0.027 | 172.6 | 1 | 0.223 | 0.016 | 227.4 | 6.2 | 0.234 | 0.004 |
| 5 | S18_5 | 99.7 | 0.8 | 0.107 | 0.01 | 104.6 | 0.9 | 0.091 | 0.019 | 116.5 | 1.9 | 0.089 | 0.012 |
| 6 | S18_6 | 79.5 | 5.2 | 0.17 | 0.017 | 94.2 | 0.5 | 0.095 | 0.011 | 103.8 | 1 | 0.074 | 0.01 |
| 7 | S19_4 | 103 | 1 | 0.118 | 0.025 | 104.3 | 0.2 | 0.125 | 0.006 | 117.7 | 1.4 | 0.128 | 0.012 |
| 8 | S19_5 | 97.8 | 4.2 | 0.163 | 0.013 | 105.3 | 0.3 | 0.085 | 0.007 | 117.7 | 2.9 | 0.096 | 0.017 |
| 9 | S19_6 | 124.3 | 2.9 | 0.137 | 0.008 | 141.5 | 0.8 | 0.085 | 0.029 | 152.4 | 1 | 0.077 | 0.023 |

The lack of significant changes in both the size and the polydispersity coefficient prove the lack of liposome aggregation, i.e. their stability in the presence of RNA-m2.

TABLE 18
Changes in the Zeta potential of series IV liposomes after the addition
of 5 μg and 10 μg RNA-m2 along with standard deviations.

| | | | | After addition of 5 μg RNA- | | | After addition of 10 μg RNA- | | |
|---|---|---|---|---|---|---|---|---|---|
| | | | | m2 per 1 mg of liposomes | | | m2 per 1 mg of liposomes | | |
| | | Before the addition | | | | Zeta | | | Zeta |
| | | of RNA-m2 | | | | potential | | | potential |
| | System | Zeta | SD | Zeta | SD | change | Zeta | SD | change |
| No. | CODE | [mV] | (+/−) | [mV] | (+/−) | [mV] | [mV] | (+/−) | [mV] |
| 1 | S17_4 | 36.67 | 1.24 | 27 | 0.42 | −9.67 | 27.37 | 1.75 | +0.37 |
| 2 | S17_5 | 36.93 | 4.89 | 29.13 | 0.32 | −7.8 | 26.17 | 3.04 | −2.97 |
| 3 | S17_6 | 47.97 | 0.58 | 37.53 | 1.19 | −10.43 | 31.03 | 2.35 | −6.5 |
| 4 | S18_4 | 47.67 | 1.31 | 42.2 | 1.92 | −5.47 | 33.23 | 0.86 | −8.97 |
| 5 | S18_5 | 49.27 | 1.45 | 44.27 | 2.47 | −5 | 31.1 | 1.78 | −13.17 |
| 6 | S18_6 | 50.9 | 1.86 | 48.9 | 1.51 | −2 | 33 | 3.86 | −15.9 |
| 7 | S19_4 | 46.4 | 2.23 | 40.6 | 0.66 | −5.8 | 33.37 | 1.43 | −7.23 |
| 8 | S19_5 | 50.73 | 3.63 | 44.37 | 2.6 | −6.37 | 38.67 | 3.15 | −5.7 |
| 9 | S19_6 | 56.07 | 2.16 | 44.3 | 0.26 | −11.77 | 32.8 | 1.82 | −11.5 |

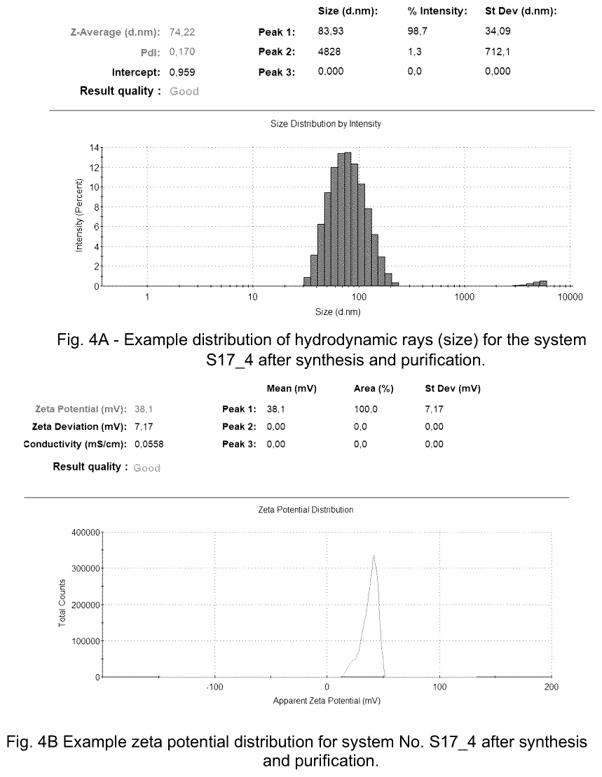

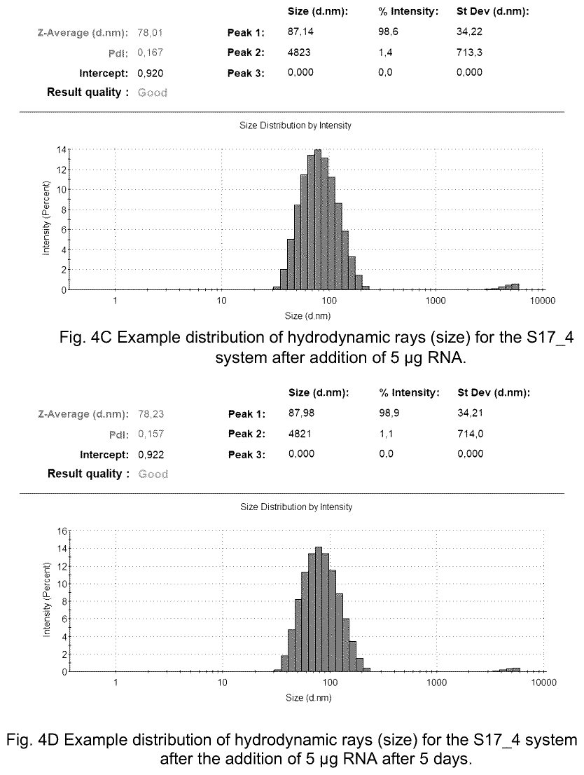

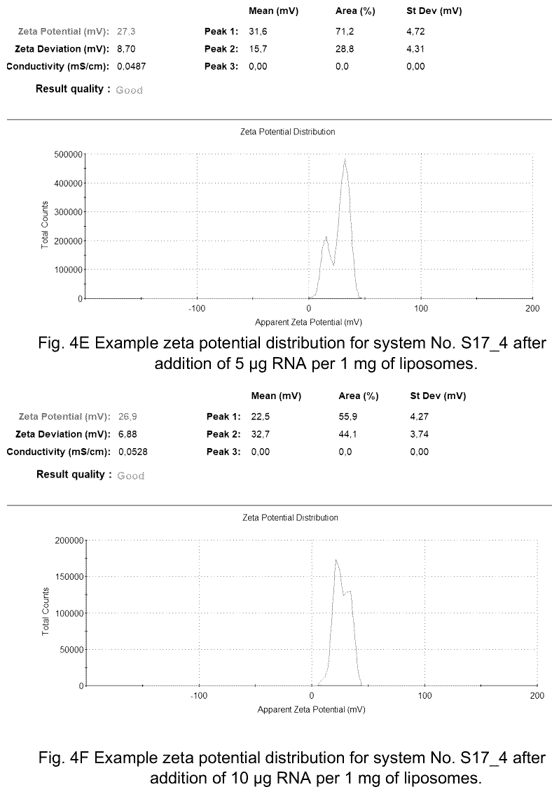

Example IV is illustrated in FIG. 4A-F

### Example V Determination of the Maximum Encapsulation Efficiency (% EE) for Liposomes From Series II (Example 2) and III (Example 3)

An encapsulation efficiency % EE, defined as the amount of drug bound in the liposome to the total amount of drug, expressed as a percentage, was determined for the liposomes in Table 1 and Table 5 loaded with emetine by UV-Vis spectroscopy. The % EE ratios for all systems exceed 90%.

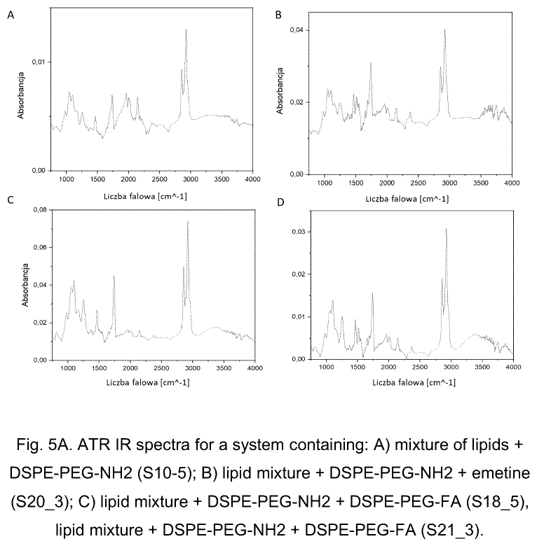

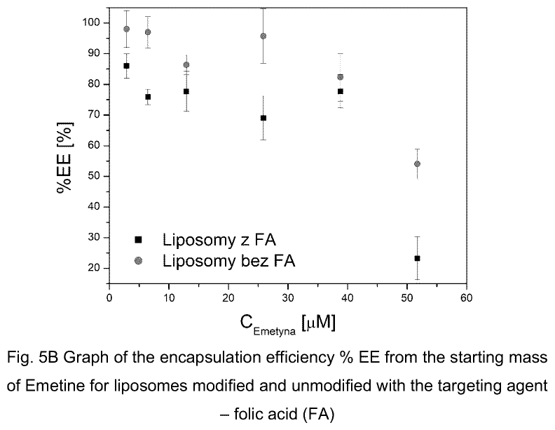

Additionally, for a more precise determination of the encapsulation efficiency, liposomes with increasing amounts of emetine were made in the range of initial concentrations of emetine from 6.5 μM to 51.7 μM for systems not modified with the targeting agent (Table 19) and modified with the targeting agent (Table 20). The physicochemical parameters along with the encapsulation efficiencies are presented in Table 21. The dependence of the encapsulation efficiency on the starting emetine concentration for the systems of Tables 19 and 20 is shown in the graph of FIG. 5B

TABLE 19
Molar and mass ratio of liposomes unmodified with folic acid targeting agent (FA) with different initial emetine content.

| | | | Molar | | | | | | | | | |
|---|---|---|---|---|---|---|---|---|---|---|---|---|
| | | | ratio of | | | | | | 14:0 | | The | |
| | Molar | | DSPE- | DSPE- | | | Molar | | EPC | 14:0 | molar | |
| System | ratio of | DPPC | PEG- | PEG_NH2 | Cholesterol | Cholesterol | ratio of | DMPC | molar | EPC | ratio of | Emetine |
| CODE | DPPC | [mg] | NH2 | [mg] | molar ratio | [mg] | DMPC | [mg] | ratio | [mg] | emetine | [mg] |
| 1S20 | 0.8 | 0.342 | 0.116 | 0.188 | 1 | 0.225 | 0.24 | 0.020 | 0.96 | 0.415 | 0.05 | 0.014 |
| 2S20 | 0.8 | 0.342 | 0.116 | 0.188 | 1 | 0.225 | 0.24 | 0.039 | 0.96 | 0.415 | 0.1 | 0.028 |
| 3S20 | 0.8 | 0.342 | 0.116 | 0.188 | 1 | 0.225 | 0.24 | 0.079 | 0.96 | 0.415 | 0.2 | 0.056 |
| 4S20 | 0.8 | 0.342 | 0.116 | 0.188 | 1 | 0.225 | 0.24 | 0.118 | 0.96 | 0.415 | 0.3 | 0.084 |
| 5S20 | 0.8 | 0.342 | 0.116 | 0.188 | 1 | 0.225 | 0.24 | 0.158 | 0.96 | 0.415 | 0.4 | 0.112 |

TABLE 20
Molar and mass ratio of liposomes modified with the targeting factor (FA) with different initial emetine content.

| | | | Molar | | | | | | | | | | Molar | |
|---|---|---|---|---|---|---|---|---|---|---|---|---|---|---|
| | | | ratio of | | Choles- | | | | 14:0 | | The | | ratio of | DSPE- |
| | Molar | | DSPE- | DSPE- | terol | Choles- | Molar | | EPC | 14:0 | molar | | DSPE- | PEG- |
| System | ratio of | DPPC | PEG- | PEG_NH2 | molar | terol | ratio of | DMPC | molar | EPC | ratio of | Emetine | PEG- | FA |
| CODE | DPPC | [mg] | NH2 | [mg] | ratio | [mg] | DMPC | [mg] | ratio | [mg] | emetine | [mg] | FA | [mg] |
| 1S21 | 0.8 | 0.342 | 0.116 | 0.188 | 1 | 0.225 | 0.24 | 0.020 | 0.96 | 0.415 | 0.05 | 0.014 | 0.01 | 0.019 |
| 2S21 | 0.8 | 0.342 | 0.116 | 0.188 | 1 | 0.225 | 0.24 | 0.039 | 0.96 | 0.415 | 0.1 | 0.028 | 0.01 | 0.019 |
| 3S21 | 0.8 | 0.342 | 0.116 | 0.188 | 1 | 0.225 | 0.24 | 0.079 | 0.96 | 0.415 | 0.2 | 0.056 | 0.01 | 0.019 |
| 4S21 | 0.8 | 0.342 | 0.116 | 0.188 | 1 | 0.225 | 0.24 | 0.118 | 0.96 | 0.415 | 0.3 | 0.084 | 0.01 | 0.019 |
| 5S21 | 0.8 | 0.342 | 0.116 | 0.188 | 1 | 0.225 | 0.24 | 0.158 | 0.96 | 0.415 | 0.4 | 0.112 | 0.01 | 0.019 |

TABLE 21
Physicochemical parameters of liposomes with different
starting mass of emetine and standard deviations (SD).

| System | | | | | | |
|---|---|---|---|---|---|---|
| CODE | Size | SD | PDI | SD | % EE | SD |
| 1S20 | 114.93 | 1.1 | 0.23 | 0.003 | 75.87 | 2.57 |
| 2S20 | 140.43 | 0.4 | 0.16 | 0.008 | 77.7 | 6.52 |
| 3S20 | 143.4 | 3.7 | 0.14 | 0.009 | 69.08 | 7.14 |
| 4S20 | 95.95 | 0.62 | 0.07 | 0.007 | 77.75 | 5.47 |
| 5S20 | 143.83 | 1.59 | 0.18 | 0.011 | 23.27 | 7.04 |
| 1S21 | 113.3 | 2.04 | 0.07 | 0.021 | 97 | 5.16 |
| 2S21 | 111 | 4.9 | 0.22 | 0.005 | 86.37 | 3.22 |
| 3S21 | 89.67 | 0.2 | 0.11 | 0.014 | 95.72 | 8.87 |
| 4S21 | 84.75 | 1.13 | 0.1 | 0.012 | 82.32 | 7.74 |
| 5S21 | 102.3 | 1.64 | 0.09 | 0.008 | 54.05 | 4.83 |

Example V is illustrated in FIG. 5A-B

### Example VI (Emetine Release Profile From S21_4 Liposomes Without and in the Presence of RNA-m2)

The 25 kD dialysis membrane was conditioned successively in: ultra pure water (Milli-Q system, Biopak CDUFBI001 filter: RNase free, DNase-free, Pyrogen-free) for 20 minutes, and Britton Robinson buffer, at appropriate pH, for another 20 minutes. Next, 1 mL of emetine-containing liposomes solution and 1 mL of a buffer with an appropriate pH were placed in the membrane. The membrane was sealed and placed in a 100 mL glass bottle with a stopper containing a buffer with a pH identical to the pH inside the dialysis membrane and a stirring device. The bottle was placed on a magnetic stirrer and sealed with aluminium foil. The amount of the released drug outside the dialysis membrane was examined using UV-Vis spectroscopy after: 10 mins, 30 mins and then every hour. The amount of emetine released was determined from the maximum of the absorbance peak at 280 nm. The measurement was carried out up to 24 h.

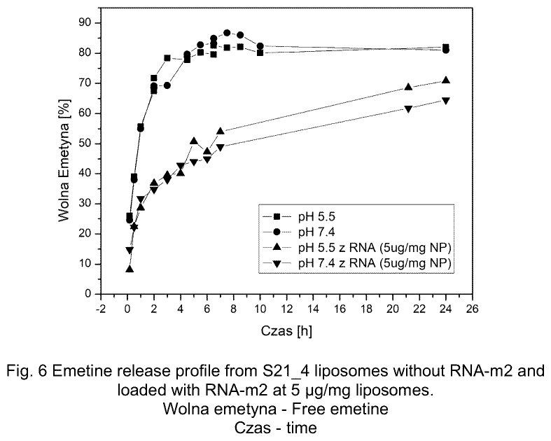

The release profiles of emetine in the absence and presence of 5 μg of RNA-m2/mg liposomes are shown in the graph of FIG. 6.

### Example VII (RNA-m2 Release Profile From S18_5 Liposomes)

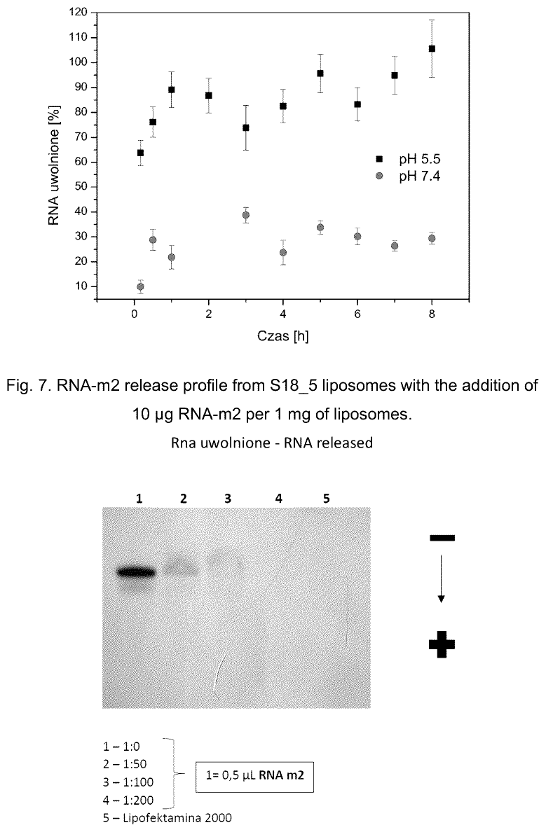

The 25 kD dialysis membrane was conditioned successively in: ultra pure water (Milli-Q system, Biopak CDUFBI001 filter: RNase free, DNase-free, Pyrogen-free) for 20 minutes, and Britton Robinson buffer, at appropriate pH, for another 20 minutes. Then, 1 mL of the S18-5 liposomes solution containing RNA-m2 of 10 μg/mg liposomes and 1 mL of buffer with appropriate pH were placed in the membrane. The membrane was sealed and placed in a 100 mL glass bottle with a stopper containing a buffer with a pH identical to the pH inside the dialysis membrane and a stirring device. The bottle was placed on a magnetic stirrer and sealed with aluminium foil. The amount of the released drug outside the dialysis membrane was examined using UV-Vis spectroscopy after: 10 mins, 30 mins and then every hour. The amount of RNA-m2 released was determined from the maximum of the absorbance peak at 250 nm. The measurement was carried out up to 8 h. The results are shown in the graph in FIG. 7.

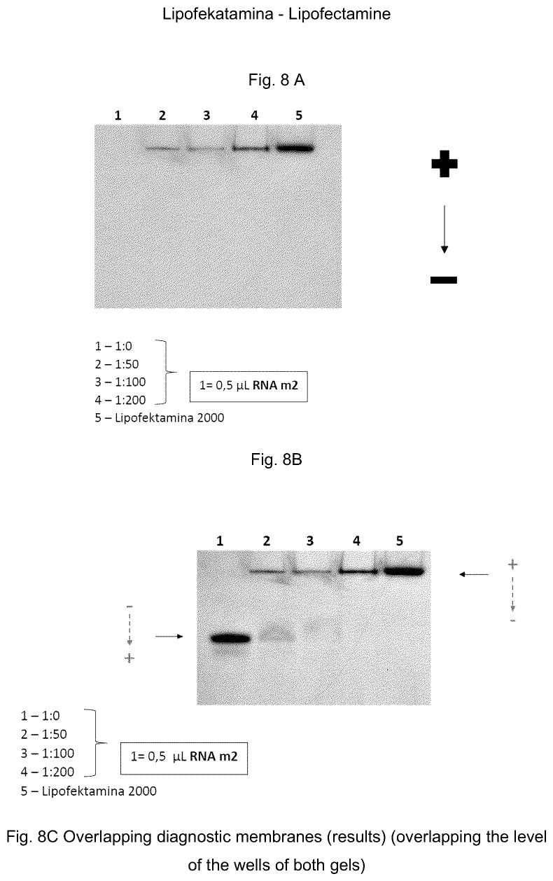

### Example VIII (Analysis of RNA-m2 Oligonucleotide Assembly With Liposomes S10_5 Without Folic Acid and Emetine, on Water by PAGE Electrophoresis)

[³²P-γ] ATP (37.0 MBq, 1.00 mCi) in a solution containing T4 polynucleotide kinase (1 L, 10,000 units/mL), 2 μL of phosphorylation reaction buffer supplied by the manufacturer, supplemented with mQ water to a volume of 20 μL were added to the RNA-m2 oligonucleotide solution (0.10 OD). The reaction solution was incubated at 37° C. for 1 hour. Then the enzyme was deactivated by incubating the mixture for 3 minutes at 80° C. Radioactively-labelled single-stranded RNA oligonucleotide (0.5 μL of the resulting solution) was incubated with the S10-5 liposomes used at the specified ratio for 1 h at room temperature (RT). Assembling was carried out in mQ water or in a buffer with the following composition: 20 mM Tris-HCl (pH 8), 50 mM NaCl and 10 mM MgCl₂. The complex formation efficiency was analysed by 15% polyacrylamide gel electrophoresis (PAGE) under non-denaturing conditions (without the addition of urea). The electrophoresis was performed at room temperature with a constant voltage of 300 V/cm and a current of 20 mA for 30 min, with specified direction of the current flow, and then the gels were autoradiographically developed. Compounds were added directly to liposomes (S10-5 dissolved in water), 1 h of incubation, RT. The results are shown in FIG. 8A-C.

### Conclusions

1. S10_5 liposomes bind nucleic acids (single strand RNA)
2. S10-5 compounds do not degrade RNA strands.
3. Gel no. 1 (−to +) In lane no. 3 you can see a band migrating slower (placed higher) than the band on lane no. 2 (placed lower), which results from the difference in the number of positively charged liposomes migrating more slowly in the gel.
4. Gel No. 2 (+to 1) RNA-coated liposomes, due to their size and/or neutral charge, poorly penetrate up to 15% of the non-denaturing gel.

### Example IX (Analysis of siRNA Oligonucleotide Assembly With Liposomes S10_5 (in Aqueous Solution Without Folic Acid and Emetine, Without NaCl) by PAGE Electrophoresis)

[³²P-γ] ATP (37.0 MBq, 1.00 mCi) in a solution containing T4 polynucleotide kinase (1 μL, 10,000 units/mL), 2 μL of phosphorylation reaction buffer supplied by the manufacturer, supplemented with mQ water to a volume of 20 μL were added to the RNA oligonucleotide solution (0.10 OD). The reaction solution was incubated at 37° C. for 1 hour. Then the enzyme was deactivated by incubating the mixture for 3 minutes at 80° C. The radioisotope-labelled RNA solution prepared as such was used in the next experiments without further purification.

Assembly of nucleic acid strand duplexes was performed by adding a solution of labelled RNA strand (sense strand) (0.10 OD) to a solution of the second strand (RNAas strand antisense or DNA-FL) (0.10 OD) at a 1:1 molar ratio in sterile water milliQ to 40 μL final volume. The mixture was heated for 5 min at 75° C., slowly cooled (1 h) to room temperature.

The radioactively labelled duplex solution (0.5 μL) was added to the S10_5 liposome solution at the specified weight ratio, incubated for 1 h at room temperature. The samples were analysed by 15% polyacrylamide gel electrophoresis (PAGE) under non-denaturing conditions (no urea added). The electrophoresis was performed at room temperature with a constant voltage of 300 V/cm and a current of 20 mA for 30 min, in a specified direction of current flow, and then the gels were developed autoradiographically on diagnostic plates. Compounds were added directly to liposomes (S10-5, no NaCl, in water), 1 h of incubation, RT.

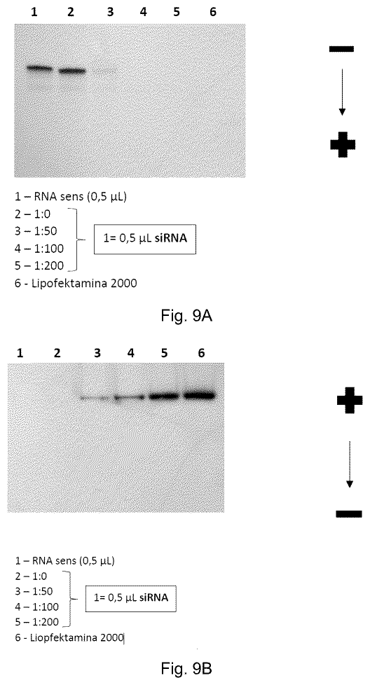

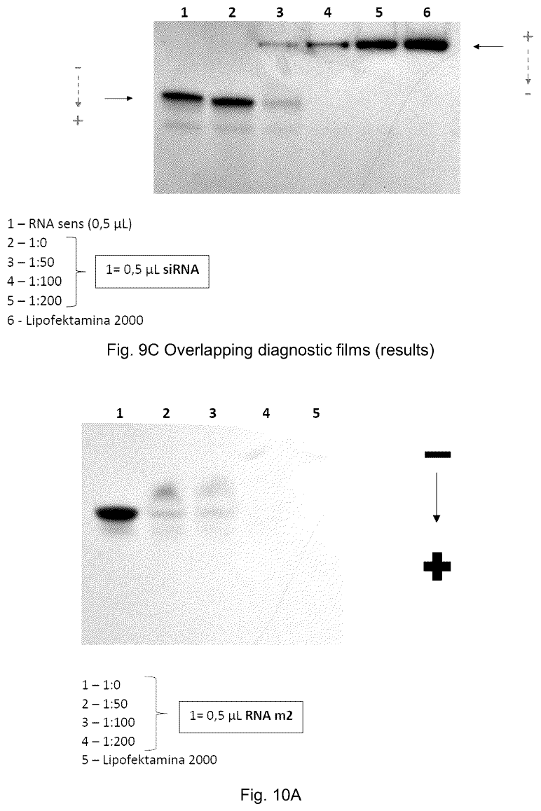

The results are shown in FIG. 9A-C.

### Conclusions

1. S10-5 liposomes without saline bind nucleic acids (siRNA duplex) better than liposomes containing NaCl.
2. Additionally, it was observed that the siRNA duplex binds more strongly to liposomes (1:50 ratio) than single-stranded RNA (1:100 ratio).
3. S10-5 compounds do not degrade siRNAs.

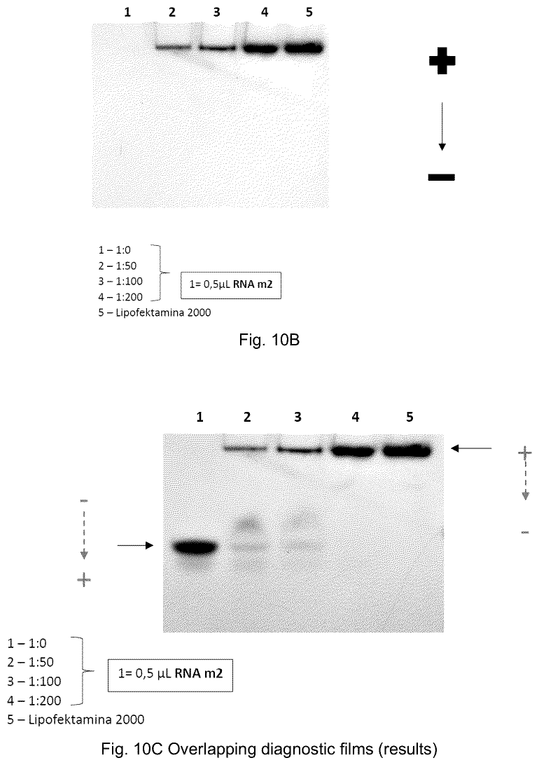

### Example X (RNA m2 Oligonucleotide Assembly Analysis With S18_2 Liposomes With Folic Acid and Emetine Without NaCl, in Water, by PAGE Electrophoresis)

[³²P-γ] ATP (37.0 MBq, 1.00 mCi) in a solution containing T4 polynucleotide kinase (1 μL, 10,000 units/mL), 2 μL of phosphorylation reaction buffer supplied by the manufacturer, supplemented with mQ water to a volume of 20 μL were added to the RNA oligonucleotide solution (0.10 OD). The reaction solution was incubated at 37° C. for 1 hour. Then the enzyme was deactivated by incubating the mixture for 3 minutes at 80° C. Radioactively-labelled single-stranded RNA oligonucleotide (0.5 μL of the resulting solution) was incubated with the S18_2 liposomes used at the specified ratio for 1 h at room temperature (RT). Assembling was carried out in mQ water or in a buffer with the following composition: 20 mM Tris-HCl (pH 8), 50 mM NaCl and 10 mM MgCl₂. The complex formation efficiency was analysed by 15% polyacrylamide gel electrophoresis (PAGE) under non-denaturing conditions (without the addition of urea). The electrophoresis was performed at room temperature with a constant voltage of 300 V/cm and a current of 20 mA for 30 min, with specified direction of the current flow, and then the gels were autoradiographically developed. Compounds (RNA m2) were added directly to emetine and folic acid liposomes (S18_2, NaCl removed, in water), 1 h of incubation, RT. The results are shown in FIG. 10A-C

### Conclusions

1. RNA m2 strongly binds to S18_2, which proves that the addition of emetine and folic acid to the liposome does not impede binding, and is stronger compared to the S10_5 liposome alone.
2. The liposome does not degrade RNA.

### Example XI (Analysis of siRNA Oligonucleotide Assembly With Liposomes S18-2 With Folic Acid and Emetine Without NaCl, in Water by PAGE Electrophoresis

[³²P-γ] ATP (37.0 MBq, 1.00 mCi) in a solution containing T4 polynucleotide kinase (1 μL, 10,000 units/mL), 2 μL of phosphorylation reaction buffer supplied by the manufacturer, supplemented with mQ water to a volume of 20 μL were added to the RNA oligonucleotide solution (0.10 OD). The reaction solution was incubated at 37° C. for 1 hour. Then the enzyme was deactivated by incubating the mixture for 3 minutes at 80° C. The radioisotope-labelled RNA solution prepared as such was used in the next experiments without further purification.

Assembly of nucleic acid strand duplexes was performed by adding a solution of labelled RNA strand (sense strand) (0.10 OD) to a solution of the second strand (RNAas strand antisense or DNA-FL) (0.10 OD) at a 1:1 molar ratio in sterile water milliQ to 40 μL final volume. The mixture was heated for 5 min at 75° C., slowly cooled (1 h) to room temperature.

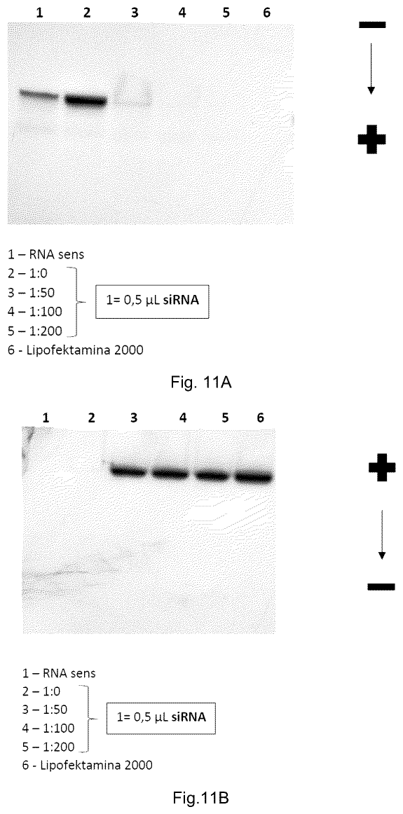

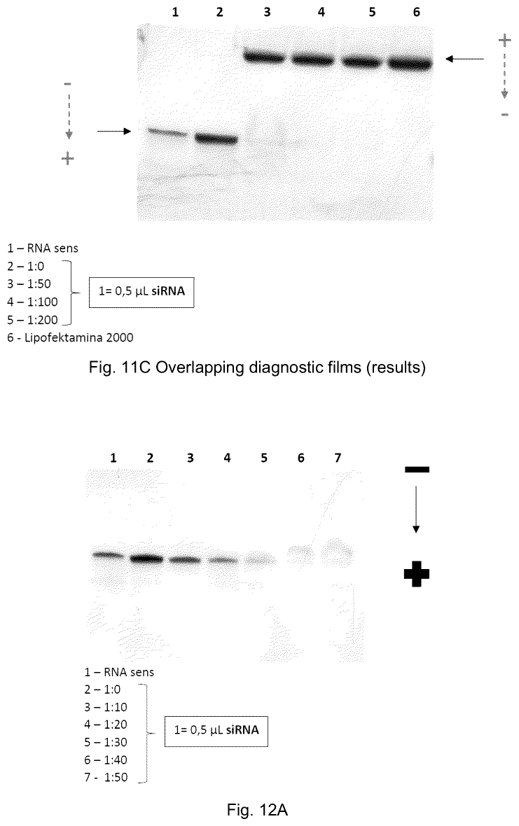

The radioactively labelled duplex solution (0.5 μL) was added to the S18_2 liposome solution at the specified weight ratio, incubated for 1 h at room temperature. The samples were analysed by 15% polyacrylamide gel electrophoresis (PAGE) under non-denaturing conditions (no urea added). The electrophoresis was performed at room temperature with a constant voltage of 300 V/cm and a current of 20 mA for 30 min, in a specified direction of current flow, and then the gels were developed autoradiographically on diagnostic plates. Compounds (siRNA) were added directly to emetine and folic acid liposomes (S18_2, NaCl removed, in water), 1 h of incubation, RT. The results are shown in FIG. 11A-C.

### Conclusions

1. Strong binding of the siRNA duplex to S18_2 was observed, despite the addition of emetine and folic acid as targeting molecule. It is due to double the amount of negative charges carried by siRNA compared to single-stranded RNA and the properties of the compound S18_2. No visible substrate (siRNA) at a ratio of 1:50 (lane No. 3).
2. The equal intensity of the bands of the formed nanostructures (+to −) is an additional confirmation of the complete saturation of S18_2 already at lower excess liposome ratios.
3. siRNA binds more strongly to S18_2 at a ratio of 1:50 than RNA m2.
4. Confirmation of S18_2 strong RNA/Liposome binding observation. The same experiment should be performed with a lower ratio of S18-2 to siRNA to confirm that the siRNA saturates the S18-2 liposome at lower ratios.

### Example XII (Analysis of the Assembly of siRNA Oligonucleotides With S18-2 Liposomes With Folic Acid and Emetine Without NaCl, in Water by PAGE Electrophoresis

[³²P-γ] ATP (37.0 MBq, 1.00 mCi) in a solution containing T4 polynucleotide kinase (1 μL, 10,000 units/mL), 2 μL of phosphorylation reaction buffer supplied by the manufacturer, supplemented with mQ water to a volume of 20 μL were added to the RNA oligonucleotide solution (0.10 OD). The reaction solution was incubated at 37° C. for 1 hour. Then the enzyme was deactivated by incubating the mixture for 3 minutes at 80° C. The radioisotope-labelled RNA solution prepared as such was used in the next experiments without further purification.

Assembly of nucleic acid strand duplexes was performed by adding a solution of labelled RNA strand (sense strand) (0.10 OD) to a solution of the second strand (RNAas strand antisense or DNA-FL) (0.10 OD) at a 1:1 molar ratio in sterile water milliQ to 40 μL final volume. The mixture was heated for 5 min at 75° C., slowly cooled (1 h) to room temperature.

The radioactively labelled duplex solution (0.5 μL) was added to the S18_2 liposome solution at the specified weight ratio, incubated for 1 h at room temperature. The samples were analysed by 15% polyacrylamide gel electrophoresis (PAGE) under non-denaturing conditions (no urea added). The electrophoresis was performed at room temperature with a constant voltage of 300 V/cm and a current of 20 mA for 30 min, in a specified direction of current flow, and then the gels were developed autoradiographically on diagnostic plates. Compounds (siRNA) were added directly to emetine and folic acid liposomes (S18_2, NaCl removed, in water), 1 h of incubation, RT.

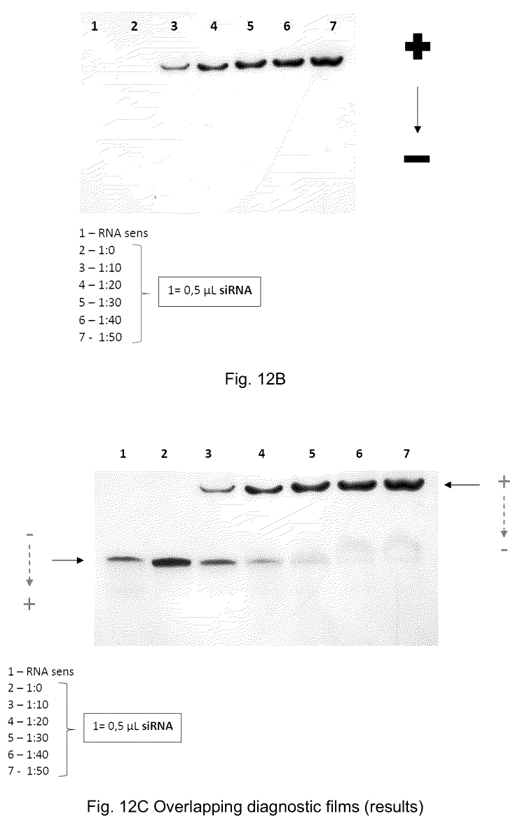

The results are shown in FIG. 12A-C

### Conclusions

1. Strong binding of the siRNA duplex to S18_2 was observed, despite the addition of emetine and folic acid as targeting molecules at a lower concentration of S18_2 liposomes. At a ratio of 1:40, the substrate (siRNA) fully bound to the S18_2 liposome (lane no. 6).

### Example XIII (Assembly of DNA-FL/RNAs (RNA-Labelled) Nucleic Acid Nanostructures With Liposomes Containing Folic Acid and Emetine (S18_2), Without NaCl, Analysis by PAGE)

Assembly of nucleic acid strand duplexes was performed by adding a solution of labelled RNA strand (sense strand) (0.10 OD) to a solution of the second strand (RNAas strand antisense or DNA-FL) (0.10 OD) at a 1:1 molar ratio in sterile water milliQ to 40 μL final volume. The mixture was heated for 5 min at 75° C., slowly cooled (1 h) to room temperature.

The radioactively labelled duplex solution (0.5 μL) was added to the S18_2 liposome solution at the specified weight ratio, incubated for 1 h at room temperature. The samples were analysed by 15% polyacrylamide gel electrophoresis (PAGE) under non-denaturing conditions (no urea added). The electrophoresis was performed at room temperature with a constant voltage of 300 V/cm and a current of 20 mA for 30 min in the specified direction of the current flow (detailed information is included in the descriptions of individual experiments), and then the gels were autoradiographically developed on diagnostic plates. Heteroduplex DNA-FL/RNAs were added directly to emetine and folic acid liposomes (S18_2, no NaCl, in water). 1 h of incubation, RT.

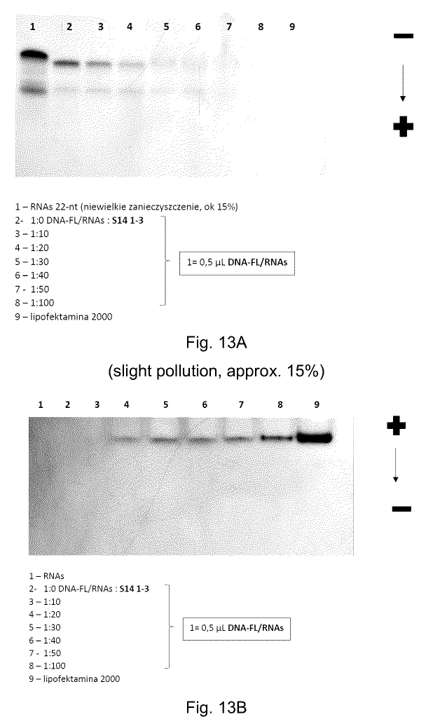

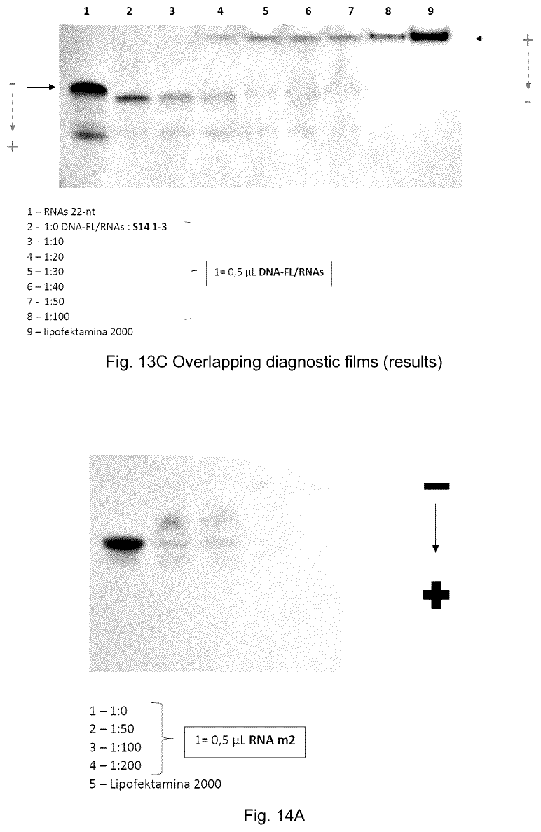

The results are shown in FIG. 13A-C.

### Conclusion

The DNA-FURNAs heteroduplex binds strongly at a ratio of 1:30-1:40. This is analogous to the siRNA homoduplex.

### Example XIV (Stability Analysis of m2 RNA Nucleic Acid Nanostructures With S10_5 Liposomes, Without NaCl After 5 Days Using PAGE Electrophoresis)

[³²P-γ] ATP (37.0 MBq, 1.00 mCi) in a solution containing T4 polynucleotide kinase (1 μL, 10,000 units/mL), 2 μL of phosphorylation reaction buffer supplied by the manufacturer, supplemented with mQ water to a volume of 20 μL were added to the RNA oligonucleotide solution (0.10 OD). The reaction solution was incubated at 37° C. for 1 hour. Then the enzyme was deactivated by incubating the mixture for 3 minutes at 80° C. The radioisotope-labelled RNA solution prepared as such was used in the next experiments without further purification.

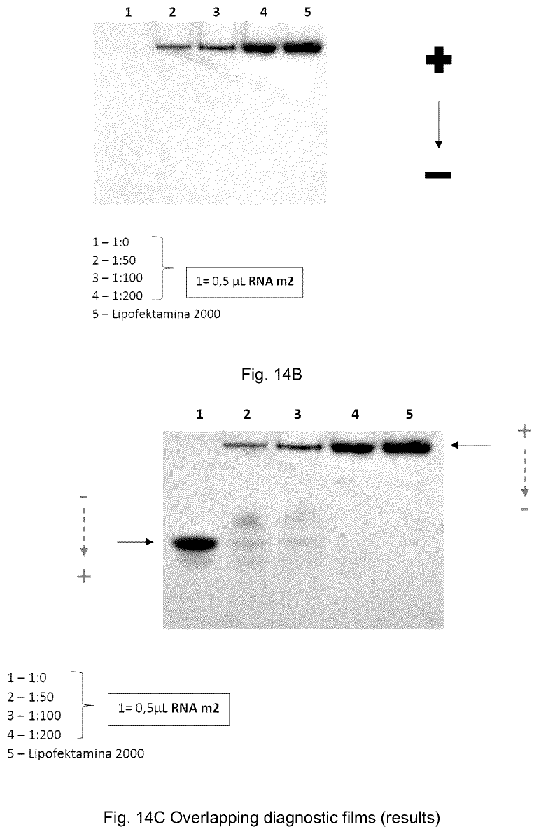

Radioactively labelled single-stranded RNA oligonucleotide (0.5 μL of the resulting solution) was incubated with the S10-5 liposomes used at the specified ratio for 1 h at room temperature (RT). Assembling was carried out in mQ water or in a buffer with the following composition: 20 mM Tris-HCl (pH 8), 50 mM NaCl and 10 mM MgCl₂. The complex formation efficiency was analysed by 15% polyacrylamide gel electrophoresis (PAGE) under non-denaturing conditions (without the addition of urea). The electrophoresis was performed at room temperature with a constant voltage of 300 V/cm and a current of 20 mA for 30 min, with specified direction of the current flow, and then the gels were autoradiographically developed. Compounds (RNA m2) added directly to liposomes without emetine and folic acid (S10-5, without NaCl, in water), 1 h of incubation, RT. The compounds were then placed in a 4° C. refrigerator for 5 days, followed by PAGE electrophoresis. The results are shown in FIG. 14A-C

### Conclusions

1. RNA m2 oligonucleotide is stable in the presence of S10-5 liposomes and does not degrade after 5 days.
2. It should also be concluded that the S10_5 liposome does not lose its properties and binds strongly nucleic acid.

### Example XV (Stability Analysis of siRNA Nucleic Acid Nanostructures With Liposomes (S18_2), Without NaCl After 3 Days by PAGE Electrophoresis)

[³²P-γ] ATP (37.0 MBq, 1.00 mCi) in a solution containing T4 polynucleotide kinase (1 μL, 10,000 units/mL), 2 μL of phosphorylation reaction buffer supplied by the manufacturer, supplemented with mQ water to a volume of 20 μL were added to the RNA oligonucleotide solution (0.10 OD). The reaction solution was incubated at 37° C. for 1 hour. Then the enzyme was deactivated by incubating the mixture for 3 minutes at 80° C. The radioisotope-labelled RNA solution prepared as such was used in the next experiments without further purification.

Assembly of nucleic acid strand duplexes was performed by adding a solution of labelled RNA strand (sense strand) (0.10 OD) to a solution of the second strand (RNAas strand antisense or DNA-FL) (0.10 OD) at a 1:1 molar ratio in sterile water milliQ to 40 μL final volume. The mixture was heated for 5 min at 75° C., slowly cooled (1 h) to room temperature.

The radioactively labelled duplex solution (0.5 μL) was added to the S18_2 liposome solution at the specified weight ratio, incubated for 1 h at room temperature. The samples were analysed by 15% polyacrylamide gel electrophoresis (PAGE) under non-denaturing conditions (no urea added). The electrophoresis was performed at room temperature with a constant voltage of 300 V/cm and a current of 20 mA for 30 min, in a specified direction of current flow, and then the gels were developed autoradiographically on diagnostic plates. Compounds (siRNA) added directly to liposomes with emetine and folic acid (S18_2, without NaCl, in water), 1 h of incubation, RT, then incubated in a refrigerator at 4° C. for 3 days.

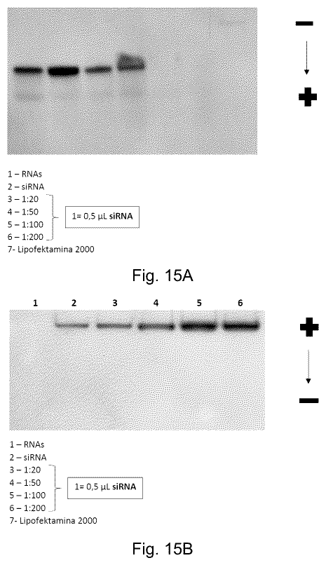

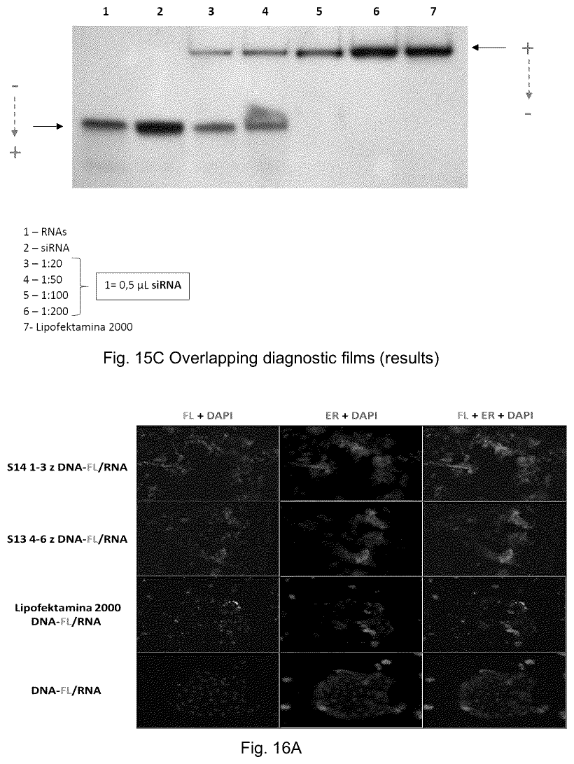

The results are shown in FIG. 15A-C.

### Conclusion

Electrophoretic analysis showed that double-stranded siRNA nucleic acids bound to S18_2 liposomes are stable after 3 days incubation in a 4° C. refrigerator

### Example XVI (Analysis of DNA-FL/RNA Penetration Capacity in Complex With Liposomes Into Caco-2 Tumour Cells and Normal MEF-WT by fluorescence microscopy.

Assembly of nucleic acid strand duplexes was performed by adding a solution of labelled RNA strand (sense strand) (0.10 OD) to a solution of the second strand (RNAas strand antisense or DNA-FL) (0.10 OD) at a 1:1 molar ratio in sterile water milliQ to 40 μL final volume. The mixture was heated for 5 min at 75° C., slowly cooled (1 h) to room temperature.

Cultivation of Caco-2 tumour cells (colorectal adenocarcinoma) and normal MEF-WT cells (mouse embryonic fibroblasts-wild-type). Conducting research on the penetration of the coated DNA-FL/RNA liposomes into cell lines with microscopic visualization.

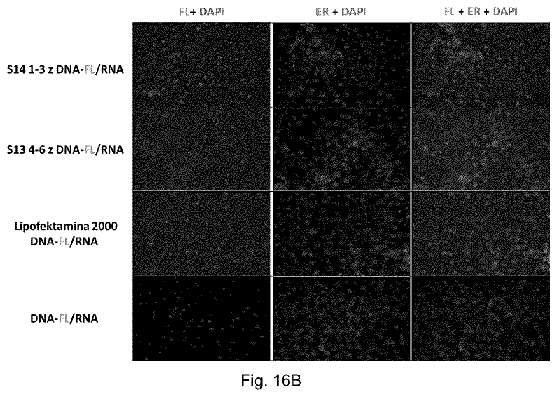

CaCo-2 and MEF-WT cells were grown according to the ATCC procedure. 15,000 cells were seeded per well of a 96-well plate in 200 μL of complete medium. After 36 h, the complete medium was removed, replacing with the stock medium, the liposome (LIPO) was added at a specific weight ratio to the nucleic acid used, i.e. 1:40, where 1=the amount of μg DNA-FL/RNA contained in 1 μL of the solution, and 40=40-fold weight excess of LIPO S18_2 (liposome with folic acid and emetine, in a salt-free aqueous solution) or 1:200, where 200=200-fold weight excess of LIPO S10_5 (liposome without folic acid and emetine, in a salt-free aqueous solution). Two control reactions were used, the first was to coat the DNA-FL/RNA duplex with a commercial transfectant, i.e. Lipofectamine 2000 (Invitrogen) at a 1:1 ratio (1 μL of Lipofectamine 2000 per 1 μg of nucleic acid), and the second using an uncoated DNA-FL/RNA heteroduplex (1 μL) without transfection agent. In each experimental well, the final reaction volume in the stock medium was 100 μL. The DNA-FL/RNA duplex was assembled analogously to siRNA without the radiolabelling process. The weak green fluorescence signal is due to the low concentrations of the DNA-FL/RNA duplex (0.1 OD DNA-FL+0.1 OD RNAs dissolved in 40 μL). Only 1 μL of duplex was used for the experiment. Microscopic photos—magnification ×20—FIG. 16A-B.

### Result

Caco-2 line (colorectal adenocarcinoma, cancer)
MEF-WT cells (mouse embryonic fibroblasts—wild-type, normal)

### Conclusions

1. DNA-FL/RNA duplex coated with S18_2 liposome (contains emetine and folic acid) strongly penetrates into Caco-2 cells, which overexpress the foil receptor on the membrane surface relative to normal MEF-WT cells.
2. The amount of DNA-FL/RNA compound taken up by the cells is shown as green fluorescence.
3. After staining the endoplasmic reticulum red and the cell nuclei blue, there is an observable (FL+ER+DAPI) change in the colour of the reticule from red to orange-yellow, which indicates the location of the DNA-FL/RNA duplex in the cytoplasm, where a large number of membranous structures are present. Additionally, therapeutic nucleic acids, e.g. (ASO, siRNA), act in the cytoplasm.
4. During the conducted experiments, it was observed that Caco-2 cells, after collecting the DNA-FL/RNA duplex coated with the S18_2 liposome, began to detach from the substrate after 4 hours, which is probably due to emetine action.
5. No similar cell changes were observed with the other systems used.
6. DNA-FL/RNA coated with S10_5 (without emetine and folic acid) also has the potential to penetrate into Caco-2 and MEF-WT cells, but much weaker than in liposome-coated S18_2 (with folic acid and emetine), but comparable and even better than the commercial Lipofectamine 2000. In this experiment, green spots (dots) are characteristic of DNA-FL/RNA transport by lipofectamine and the S10_2 liposome delivers them all over the surface in the form of “green cloud” green shadows.
7. MEF-WT cells are less transfected with Lipofectamine 2000 than cancerous Caco-2 cells, which is a characteristic feature of a given cell line.

### Example XVII

Anti-EGFR siRNA compounds were added directly to the lipoplex and incubated for 0.5 h. At a ratio of 1:10, the substrate (anti-EGFR siRNA), the substrate fully bound to the lipoplex. The study verified the release of anti-EGFR siRNA from the lipoplex after 30 minutes in various pH ranges.

1. A cationic liposome that binds and stabilises RNA, characterised in that it consists of neutral lipids in the amount of 12.4 to 49% by weight, cationic lipids in the amount of 16.2 to 55% by weight, polyethylene glycol-modified lipids in the amount of 12.9 to 15.1% by weight and cholesterol in an amount from 15.4 to 18.1% by weight, and is characterised by a size from 80 nm to 190 nm, a polydispersity index from 0.06 to 0.23, and a zeta potential from +19 mV to +55 mV, wherein it is loaded with RNA.

2. Liposome according to claim 1, characterised in that the RNA is an anti-EGFR siRNA duplex.

3. Liposome according to claim 1, characterised in that dipalmitoylphosphatidylcholine (DPPC) is the neutral lipid.

4. Liposome according to claim 1, characterised in that 1,2-dimyristoyl-sn-glycero-3-phosphocholine (DMPC) is the neutral lipid.

5. Liposome according to claim 1, characterised in that 1,2-dimyristoyl-sn-glycero-3-ethylphosphocholine (14:0 EPC) is the cationic lipid.

6. Liposome according to claim 1, characterised in that 1,2-dipalmitoyl-sn-glycero-3-ethylphosphocholine (16:0 EPC) is the cationic lipid.

7. Liposome according to claim 1, characterised in that 1,2-distearoyl-sn-glycero-3-phosphoethanolamine-N-[amino (polyethylene glycol)-2000] (DSPE-PEG-NH₂) is the polyethylene glycol-modified lipid.

8. Liposome according to claim 1, characterised in that the RNA is linked to the liposomes in the proportion of 1:30, that is, 1 mg of liposome contains no more than 1.34 μg of RNA.

9. Liposome according to claim 1, characterised in that the low molecular weight drug is embedded in it.

10. Liposome according to claim 9, characterised in that emetine is the low molecular weight drug in an amount of not less than 0.5% by weight and not more than 8.5% by weight.

11. The liposome according to claim 1, characterised in that it is modified with a targeting agent.

12. Liposome according to claim 11, characterised in that folic acid is the targeting agent in an amount of not less than 1.3% by weight and not more than 1.55% by weight.

13. The use of a liposome as described in claim 1 as a drug carrier.

14. The use of a liposome according to claim 13 in gene therapy.

15. The use of a liposome according to claim 13 in antiviral therapy.

16. The use of a liposome according to claim 13 in cancer therapy.

17. The method of loading the liposome with emetine, characterised in that from 12.4 to 49% by weight of neutral lipids, from 16.2 to 55% by weight of cationic lipids, from 12.9 to 15.1% by weight of polyethylene glycol-modified lipids and cholesterol in the amount of from 15.4 to 18.1% by weight and not less than 0.5% by weight of emetine are weighted, then the sample is dissolved in a 2:1 mixture of chloroform and methanol by adding 0.26 to 1.4 mg of the lipid mixture for each 1 mL of the solvent mixture, then the entirety is evaporated by removing the solvents at a temperature from 25 to 40° C., at a pressure from 100 to 300 mbar for 2 to 5 hours until a thin film of the lipid bilayer is obtained on the vessel walls, and then post-dried in temperature from 25° C. to 40° C. and pressure from 100 to 300 mbar; after complete evaporation of organic solvents, sterile filtered saline, PBS buffer or Ringer's solution in the amount of 1 mL per 0.34 to 0.36 mg of lipids is added, tightly closed and then hydrated at a temperature from 45 to 60° C., at a pressure from 100 to 300 mbar for 5 to 10 hours, the hydrated lipid layers are left to cool down, and then the lipids are broken down into particles with a size not exceeding 200 nm at a temperature of 45 to 65° C., monitoring the particle size and the amount of bound emetine, the solution is allowed to cool, finally the obtained liposomes are purified from unbound emetine and unreacted lipids, the Zeta potential is measured and the composition is characterised.

18. The method according to claim 17, characterised in that dipalmitoylphosphatidylcholine (DPPC) is the neutral lipid.

19. The method according to claim 17, characterised in that 1,2-dimyristoyl-sn-glycero-3-phosphocholine (DMPC) is the neutral lipid.

20. The method according to claim 17, characterised in that 1,2-dimyristoyl-sn-glycero-3-ethylphosphocholine (14:0 EPC) is the cationic lipid.

21. The method according to claim 17, characterised in that 1,2-dipalmitoyl-sn-glycero-3-ethylphosphocholine (16:0 EPC) is the cationic lipid.

22. The method according to claim 17, characterised in that 1,2-distearoyl-sn-glycero-3-phosphoethanolamine-N-[amino (polyethylene glycol)-2000] (DSPE-PEG-NH₂) is the polyethylene glycol-modified lipid.

23. The method according to claim 17, characterised in that emetine is obtained as follows: a solution of emetine hydrochloride in deionised water is prepared at a concentration of 30 mg/mL, then a precipitate is precipitated by titration with 0.2 M NaOH solution, in the amount of 1 mL 0.2 M NaOH for every 20 mg of emetine, then the precipitate is centrifuged at 4000 to 6000 RPM for 10 to 30 minutes and separated from the solution, then 0.1 mL of 0.2M NaOH is added to the supernatant solution to control complete precipitation of emetine, and the amount of unprecipitated emetine is measured, and the sediment is washed not less than five times with distilled water, each time dispersing with ultrasounds, centrifuging and measuring the amount of unprecipitated emetine in the supernatant solution, the total loss of emetine during the process is not more than 20%, then the sediment is post-dried at a temperature of 25 to 30° C. and a pressure of 100 mbar for 3 to 5 hours.

24. The method according to claim 23, characterised in that the amount of unprecipitated emetine is measured by UV-Vis spectroscopy against a calibration curve.

25. The method according to claim 17, characterised in that the lipid mixture is evaporated in a vacuum dryer at a temperature from 25 to 40° C. and a pressure from 100 to 300 mbar for 2 to 4.5 h.

26. The method according to claim 17, characterised in that the lipid mixture is evaporated in a vacuum evaporator at a temperature from 30 to 40° C. and a pressure from 150 to 300 mbar for 2 to 5 hours.

27. The method according to claim 17, characterised in that the hydration is performed in a vacuum dryer.

28. The method according to claim 17, characterised in that the hydration is performed in a vacuum evaporator.

29. The method according to claim 17, characterised in that the post-drying is carried out in a vacuum dryer.

30. The method according to claim 17, characterised in that sterile saline, PBS buffer, or Ringer's solution is filtered through bpPVDF, 0.2 μm syringe filters.

31. The method according to claim 17, characterised in that the breakdown of lipids is carried out by sonication for 30 to 60 min at a frequency of 50 Hz until the colour of the solution changes from milky to colourless.

32. The method according to claim 17, characterised in that the amount of bound emetine is monitored by the UV-Vis method at a wavelength of 280 nm.

33. The method according to claim 17, characterised in that the liposomes are purified by dialysis on 10 kD membranes with ultrapure water for 30 to 60 minutes by administering 1 litre of water to 4.5 mL of the sample, and changing the water three times at regular intervals.

34. The method according to claim 17, characterised in that the purity of the obtained liposomes is measured by UV-Vis spectroscopy.

35. The method according to claim 17, characterised in that the size of the obtained liposomes is determined by the DLS method.

36. The method according to claim 17, characterised in that the characterization of the composition of the liposomes is performed by the ATR IR method.
